(19) United States
(12) Patent Application Publication
(10) Pub. No.: US 2011/0139885 A1
(43) Pub. Date: Jun. 16, 2011

(54) REFILL, WICK ASSEMBLY FOR USE WITH A REFILL, AND METHOD OF RETAINING A REFILL

(76) Inventors: THOMAS P. GASPER, Germantown, WI (US); Chris A. Kubicek, East Troy, WI (US); Andrew N. Niemiec, Franklin, WI (US); Steven V. Bisbikis, Hawthorn Woods, IL (US); Jason R. Gebhardt, Buffalo Grove, IL (US)

(21) Appl. No.: 12/969,261

(22) Filed: Dec. 15, 2010

Related U.S. Application Data
(60) Provisional application No. 61/286,575, filed on Dec. 15, 2009.

(51) Int. Cl. A61L 9/12

(52) U.S. Cl. 239/6; 239/44

(57) ABSTRACT

A volatile material refill includes a container with a volatile material disposed therein, a plug assembly disposed within an opening of the container and adapted to retain a wick extending out of the opening, a sheath covering a portion the wick, and at least one rib extending outwardly from the sheath and adapted to assist in retaining the refill in at least one volatile material dispenser.

### CROSS REFERENCE TO RELATED APPLICATIONS

This application claims the benefit of U.S. Provisional Patent Application No. 61/286,575, filed Dec. 15, 2009, which is hereby incorporated by reference herein.

### REFERENCE REGARDING FEDERALLY SPONSORED RESEARCH OR DEVELOPMENT

Not applicable

### SEQUENTIAL LISTING

Not applicable

### BACKGROUND OF THE INVENTION

1. Field of the Invention

The present invention relates generally to refills for use with volatile material dispensers, and more particularly, to refills for use in a plurality of differently dimensioned volatile material dispensers.

2. Description of the Background of the Invention

Multiple different volatile material dispensers are commercially sold and generally include a housing and a refill that is inserted into the housing. The refill generally includes a container for holding a volatile material therein. In some dispensers, the volatile material is passively emitted therefrom. In other dispensers, a diffusion element is utilized to facilitate the dispensing of the volatile material. Examples of diffusion elements include heaters, piezoelectric elements, fans, aerosol actuators, and the like. Regardless of the manner in which the volatile material is emitted, once the volatile material has been expended from the refill, the refill is removed by a user and replaced with a new refill.

One type of commercial volatile material dispenser, referred to herein as a plug-in scented oil dispenser, includes a housing and a heater disposed within the housing. A refill for use with a plug-in scented oil dispenser generally includes a container portion having a bottom end and a top end, wherein the container portion terminates in a neck portion at the top end. A volatile material is disposed within the container portion and a wick is in contact with the volatile material and extends out of the refill through the neck portion. A plug or other connector generally positions and retains the wick within the neck. Upon insertion of the refill into the dispenser, at least a portion of the wick is disposed adjacent the heater such that volatile material that moves through the wick is volatilized by the heater.

One of the disadvantages of many of the volatile material dispensers and refills therefore, such as the plug-in scented oil dispenser discussed above, is that each refill has features that are unique or complementary to the particular dimensions of the housing of the dispenser for which it is sold. Still further, each type of dispenser is adapted to accept only a single type of refill having features unique or complementary to that dispenser. For example, all plug-in scented oil dispensers sold by S. C. Johnson & Son, Inc. (“S. C. Johnson”) of Racine, Wis., generally only accept refills sold by S. C. Johnson under their Glade® brand and such refills are designed to only fit within S. C. Johnson plug-in scented oil dispensers. Similarly, all plug-in scented oil dispensers sold by Reckitt Benckiser (“Reckitt”) of England generally only accept refills sold by Reckitt under their Air Wick® brand and such refills are designed to only fit within Reckitt plug-in scented oil dispensers. This is generally the case for most commercial plug-in scented oil dispensers and refills therefore. This can cause problems with the consumer, because consumers may forget the brand of dispenser they have and/or have multiple dispensers in their home and cannot recall which dispenser needs to be refilled. When a consumer buys an incorrect refill, the consumer oftentimes becomes aggravated.

In recent years, attempts have been made to create a universal refill such that a single refill may interfit with housings of volatile material dispensers sold by multiple different companies under different brands. For example, a universal refill includes a bottle with first and second opposing external surfaces and third and fourth opposing external surfaces. The first external surface is arcuate and a pair of projections extend outwardly from the third and fourth external surfaces. A neck extends upwardly from an upper surface of the bottle and includes an opening therethrough. An annular collar is formed integrally with the neck. A centerline of the opening is coincident with a midpoint between the third and fourth external surfaces and the centerline of the opening is offset from a midpoint between the first and second external surfaces. The universal refill is capable of being inserted into volatile material dispensers sold by S. C. Johnson under the Glade® brand and volatile material dispensers sold by Reckitt under the Air Wick® brand, which are both discussed in more detail hereinafter. In particular, the universal refill is inserted into the Glade® dispenser such that the third and fourth external surfaces of the refill are disposed adjacent front and rear walls of the Glade® dispenser. Once the refill is inserted into the Glade® dispenser, the projections on the third and fourth external surfaces are retained within a groove and aperture in the rear and front walls, respectively, of the Glade® dispenser. To insert the universal refill in the Air Wick® dispenser, the refill is rotated 90 degrees such that the first external surface is facing a rear wall of the Air Wick® dispenser. As the refill is inserted into the Air Wick® dispenser, the arcuate pattern on the first external surface guides the refill into the Air Wick® dispenser such that latches within the Air Wick® dispenser grasp onto the collar of the refill and retain the refill within the dispenser. Such complicated methods of insertion for different dispensers can be confusing to the consumer, oftentimes leading to incorrect insertion of the refill. A further universal refill has been created wherein the refill has a neck portion that is offset between front and rear walls of the refill, but such refill has inherent problems, such as, difficulty in manufacturing, difficulty in filling, and complications inserting the refill into dispensers having symmetrical dimensions.

In another instance, an adapter for a refill has been developed, wherein the refill alone is capable of insertion into a first housing, but not a second differently dimensioned housing. The refill includes a container for holding a volatile material, a neck extending upwardly from the container, and a wick disposed within the container and extending out the neck. An adapter may be added to the refill such that the refill with the adapter thereon may be inserted into the second housing, but not the first housing, when the adapter is affixed to the refill. The adapter includes a means for attaching the adapter to a refill, wherein the means for attaching may include, for example, a neck portion that may be placed around the neck of the refill, an adhesive, and/or a snap fit about the container or the neck of the refill. The adapter further includes means for altering a shape of the container, wherein the means for altering may include, for example, projections, grooves, and/or the like. The main purpose of the adapter is to provide added girth to the container of a refill that would otherwise not fit within housings having larger dimensions.

### SUMMARY

According to one aspect of the present invention, a volatile material refill includes a container with a volatile material disposed therein, a plug assembly disposed within an opening of the container, and a wick retained in the container by the plug assembly. The refill further includes a sheath covering a portion the wick and at least one rib extending outwardly from the sheath and adapted to assist in retaining the refill in at least one volatile material dispenser.

In a different aspect of the present invention, a wick assembly for use in a refill comprises a plug assembly adapted to be inserted into a container of a refill, a sheath extending from the plug assembly, and at least one surface projection extending outwardly from the sheath and adapted to be disposed within a channel in one or more volatile material dispensers.

In a further aspect of the present invention, a method of retaining a refill in a volatile material dispenser includes the steps of providing a container having an elongate wick retained within the container by a plug assembly and providing a sheath having at least two surface projections extending outwardly therefrom, wherein the sheath is disposed around at least a portion of the wick. The method further includes the step allowing for positioning of the wick in a channel within the volatile material dispenser such that the surface projections contact at least one surface forming the channel.

### BRIEF DESCRIPTION OF THE DRAWINGS

FIG. 1 is a top isometric view of a prior art refill;

FIG. 2A is an exploded view of the refill of FIG. 1;

FIG. 2B is a cross-section view of the refill of FIG. 1 taken generally along the lines 2B-2B of FIG. 1;

FIG. 2C is a front elevational view of the refill of FIG. 1;

FIG. 3 is a top isometric view of a first volatile material dispenser;

FIG. 4 is a top isometric view of a second volatile material dispenser;

FIG. 4A is a cross-section view of the dispenser of FIG. 4 taken generally along the lines 4A-4A of FIG. 4;

FIG. 5 is a top isometric view of a first embodiment of a refill of the present invention;

FIG. 6 is a cross-sectional view of a wick, sheath, and ribs of the refill of FIG. 5 and taken generally along the lines 6-6 of FIG. 5;

FIG. 7 is cross-sectional view of the refill of FIG. 5 (taken generally along the lines 7-7) inserted into the volatile material dispenser of FIG. 3 and taken generally along the lines 7-7 of FIG. 3;

FIG. 8 is a cross-sectional view of the refill of FIG. 5 (taken generally along the lines 7-7) inserted into the volatile material dispenser of FIG. 4 and taken generally along the lines 8-8 of FIG. 4;

FIG. 8A is a cross-sectional view of a further refill inserted into the volatile material dispenser of FIG. 4 and taken generally along the lines 8-8 of FIG. 4;

FIG. 9A is a top isometric view of a second embodiment of a refill of the present invention;

FIG. 9B is a cross-sectional view of a wick, sheath, ribs, and adapter of the refill of FIG. 9A and taken generally along the lines 9B-9B of FIG. 9A;

FIG. 10 is a cross-sectional view of the refill of FIG. 9A (taken generally along the lines 10-10) inserted into the volatile material dispenser of FIG. 3 and taken generally along the lines 7-7 of FIG. 3;

FIG. 11 is a cross-sectional view of the refill of FIG. 9A (taken generally along the lines 10-10) inserted into the volatile material dispenser of FIG. 4 and taken generally along the lines 8-8 of FIG. 4;

FIG. 12 is a cross-sectional view similar to the view of FIG. 11 of a third embodiment of a refill of the present invention similar to the embodiment of FIG. 9A;

FIG. 13 is a top isometric view of a fourth embodiment of a refill of the present invention;

FIG. 14 is a cross-sectional view of the refill of FIG. 13 (taken generally along the lines 14-14) inserted into the volatile material dispenser of FIG. 3 and taken generally along the lines 7-7 of FIG. 3;

FIG. 15A is a cross-sectional view of the refill of FIG. 13 (taken generally along the lines 14-14) inserted into the volatile material dispenser of FIG. 4 and taken generally along the lines 8-8 of FIG. 4;

FIG. 15B is a cross-sectional view of the refill of FIG. 13 (taken generally along the lines 14-14) inserted into the volatile material dispenser of FIG. 4 and taken generally along the lines 4A-4A of FIG. 4;

FIG. 15C is a cross-sectional view similar to that of FIG. 15B showing a variation of the embodiment of FIGS. 12-15B;

FIG. 16 is top isometric view of a fifth embodiment of a refill of the present invention with an attachment affixed thereto;

FIG. 17A is a top isometric view of the attachment of FIG. 16 removed from the refill thereof;

FIG. 17B is a side elevational view of the attachment of FIG. 16 removed from the refill thereof;

FIG. 18 is a cross-sectional view of the refill of FIG. 16 (taken generally along the lines 18-18) inserted into the volatile material dispenser of FIG. 4 and taken generally along the lines 8-8 of FIG. 4;

FIG. 19 is a cross-sectional view of the refill of FIG. 16 (taken generally along the lines 18-18) inserted into the volatile material dispenser of FIG. 4 and taken generally along the lines 4A-4A of FIG. 4;

FIG. 20 is a front elevational view of a sixth embodiment of a refill of the present invention with an attachment affixed thereto;

FIG. 21 is a top isometric view of an attachment of FIG. 20 removed from the refill thereof;

FIG. 22 is a top isometric view of a seventh embodiment of a refill of the present invention;

FIG. 23 is a front elevational exploded view of the refill of FIG. 22;

FIG. 24 is a cross-sectional view of the refill of FIG. 22 (taken generally along the lines 24-24) inserted into the volatile material dispenser of FIG. 4 and taken generally along the lines 8-8 of FIG. 4;

FIG. 25 is a top isometric view of an eighth embodiment of a refill of the present invention;

FIG. 26 is a cross-sectional view of the refill of FIG. 25 (taken generally along the lines 26-26) inserted into the volatile material dispenser of FIG. 3 and taken generally along the lines 7-7 of FIG. 3;

FIG. 27 is a cross-sectional view of the refill of FIG. 25 (taken generally along the lines 26-26) inserted into the volatile material dispenser of FIG. 4 and taken generally along the lines 8-8 of FIG. 4;

FIG. 28 is a top isometric view of a ninth embodiment of a refill of the present invention;

FIG. 29 is a cross-sectional view of the refill of FIG. 28 (taken generally along the lines 29-29) inserted into the volatile material dispenser of FIG. 3 and taken generally along the lines 7-7 of FIG. 3; and

FIG. 30 is a cross-sectional view of the refill of FIG. 28 (taken generally along the lines 30-30) inserted into the volatile material dispenser of FIG. 4 and taken generally along the lines 8-8 of FIG. 4.

Other aspects and advantages of the present invention will become apparent upon consideration of the following detailed description, wherein similar structures have like or similar reference numerals.

### DETAILED DESCRIPTION

The present invention is directed to refills and methods for inserting such refills into a plurality of volatile material dispensers. While the present invention may be embodied in many different forms, several specific embodiments are discussed herein with the understanding that the present invention is to be considered only as an exemplification of the principles of the invention, and it is not intended to limit the invention to the embodiments illustrated.

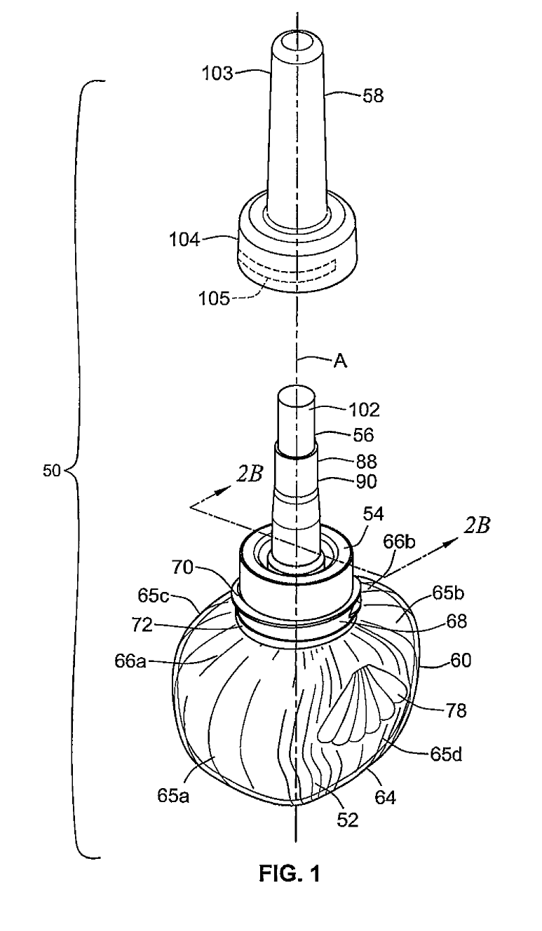

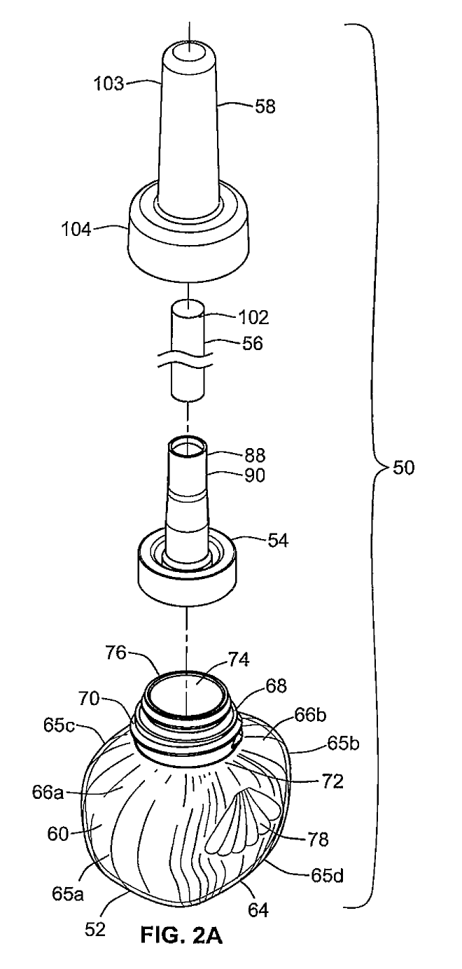

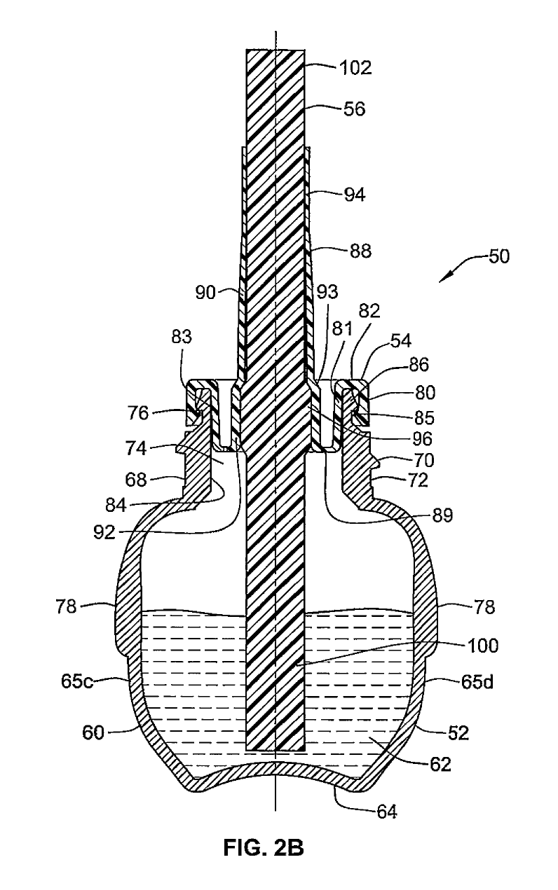

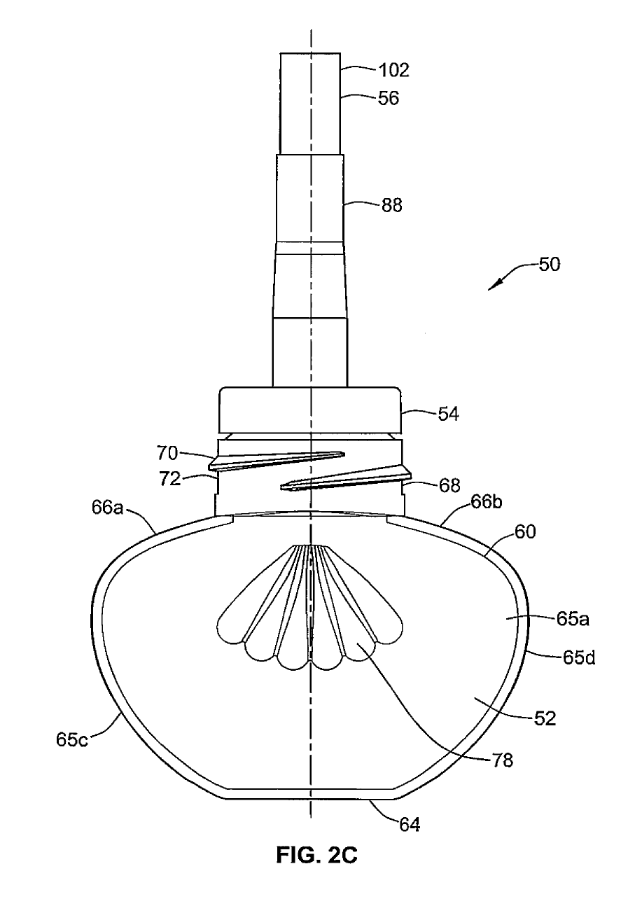

Referring to the drawings, FIGS. 1, 2A, 2B, and 2C depict a commercial refill 50 that includes a container 52, a plug assembly 54 to hold a wick 56 within the container 52, and a removable cap 58 to cover the wick 56 prior to use. The container 52 includes a body 60 with a volatile material 62 disposed therein. The body 60 includes a base portion 64, first and second opposing walls 65a, 65b that extend upwardly and curve outwardly before curving inwardly at first and second top walls 66a, 66b that are integral with a neck 68, and third and fourth opposing walls 65c, 65d that extend upwardly in a generally vertical manner and curve inwardly toward the neck 68. As can be seen in FIGS. 1, 2A, 2B, and 2C the refill 50 is symmetrical about the neck 68 in that the first and second walls 65a, 65b are disposed equal distances from an axis A of the refill 50 and the third and fourth walls 65c, 65d are disposed equal distances from the axis A. Additionally, all other features of the refill 50 are symmetrical about the axis A. The neck 68 includes a threaded portion 70 disposed on an outer surface 72 thereof and an opening 74 disposed through a top portion 76 thereof, wherein the opening 74 allows access to the volatile material 62. The container 52 further includes raised portions 78 extending outwardly from the third and fourth opposing walls 65c, 65d, wherein the raised portions 78 are in the form of inverted shell-shaped members. The raised portions 78 may alternatively be in the form of trade indicia or another shape that cooperates with a housing for insertion of the refill 50 therein, as discussed in greater detail hereinafter.

The volatile material 62 disposed in the container 52 may be of any type of volatile material adapted to be dispensed into the air. For example, the container 52 may include a cleaner, an insecticide, an insect repellant, an insect attractant, a disinfectant, a mold or mildew inhibitor, a fragrance, a disinfectant, an air purifier, an aromatherapy scent, an antiseptic, an odor eliminator, a positive fragrancing volatile material, an air-freshener, a deodorizer, or the like, and combinations thereof. Additives may be included in the volatile material, such as, for example, fragrances, and/or preservatives.

Still referring to FIGS. 1, 2A, 2B, and 2C the plug assembly 54 is generally rigid and includes an outer annular ring 80 and an inner annular ring 81 that are generally vertical and connected by a generally horizontal surface 82. The inner annular ring 81 is designed to have an outer surface 83 that is complementary to and abuts an inner surface 84 forming the opening 74 of the container 52. The outer annular ring 80 includes an inwardly extending annular projection 85 for snapping onto an outwardly extending annular projection 86 on the neck 68 of the container 52 to retain the plug assembly 54 on the container 52. The plug assembly 54 further includes a generally rigid sheath 88 having an inwardly extending annular portion 89 and an upwardly extending cylindrical portion 80. The annular portion 89 and the cylindrical portion 90 surround the wick 56. The cylindrical portion 90 includes a lower portion 92, a tapered central portion 93, and an upper portion 94, wherein the lower portion 92 has a greater diameter than the upper portion 94. An annular projection 96 formed on the wick 56 interferes with the tapered central portion 93 due to the difference in diameters of the lower and upper portions 92, 94 to prevent removal of the wick 56 from the plug assembly 54 and controls vertical position of wick 56 in sheath 88.

The wick 56 is elongate such that a bottom portion 100 of the wick 56 extends into contact with and absorbs the volatile material 62 disposed within the container 52. As noted above, the annular projection 96 formed on the wick 56 is disposed within the plug assembly 54 and a top portion 102 of the wick 56 extends upwardly from the plug assembly 54 and is exposed to the ambient environment. At least a segment of the top portion 102 of the wick 56 is surrounded by the upper portion 94 of the sheath 88 to protect the wick 56 from heat that is applied thereto. The amount of surface area of the wick 56 protruding beyond the sheath 88 results in an exposed are, which affects the resulting fragrance delivery rate. The wick 56 is depicted as a substantially uniform cylinder with a substantially constant diameter throughout the entire length thereof, except at the annular projection 96. However, the wick 56 may be other shapes and sizes as known in the art. The wick 56 may be made of one material or alternatively may be made of multiple materials, for example, the bottom portion 110 of the wick 56 may be made of a first material and the top portion 102 of the wick 56 may be made of a second material. Materials suitable for the wick 56 include any polymeric or porous materials and/or any other materials known in the art that are suitable for wicking.

The removable cap 58 is disposed over the wick 56 for shipping and storage of the refill 50. An upper generally cylindrical portion 103 of the cap 58 covers the wick 56 and a lower generally cylindrical portion 104 has a shape complementary to the neck 68 of the container 62. The cap 58 includes a threaded portion 105 on an internal surface 106 thereof for mating with the threaded portion 70 of the neck 68 to secure the cap 58 to the refill 50. By way of the threaded portions 70, 105, the removable cap 58 may be repeatedly removed from and replaced on the neck 68 of the container 52, if desired by a user. When the removable cap 58 is secured to the neck 68, the wick 56 is not exposed to the environment and no volatile active 62 is allowed to escape. In use, the user may remove the cap 58 by twisting, which will result in the wick 56 being exposed to the environment. The cap 58 is optional and may be secured to the container 52 through other methods as known in the art and as disclosed herein.

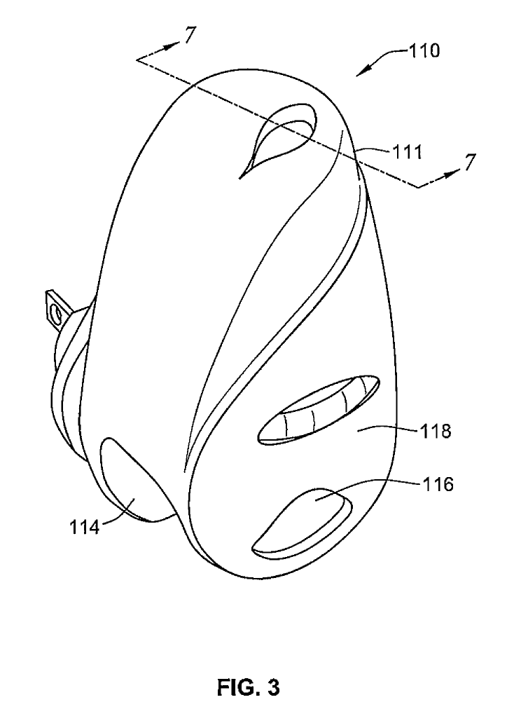

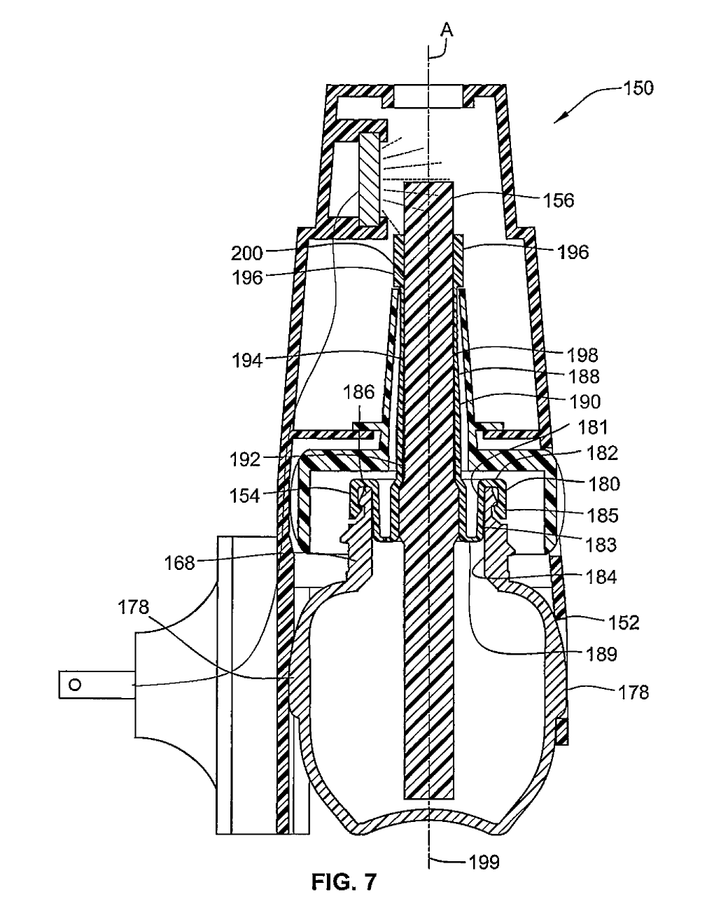

When the cap 58 is removed, the refill 50 may be inserted into and used with a first commercial dispenser 110 shown in FIG. 3. The dispenser 110 includes a housing 111 and a heater (not shown) disposed within the housing 111. The refill 50 is inserted into the housing 111 by inserting the wick 56 thereof into a channel (for example, channel 210 shown in FIG. 7) disposed adjacent the heater. Once the wick 56 is aligned within the channel, the refill 50 is moved upwardly into the dispenser 110 until the raised portions 78 extending outwardly from the third and fourth opposing walls 65c, 65d of the container 52 slide into and are retained within a groove 112 formed in a rear wall 114 of the housing 111 and an aperture 116 disposed within a front wall 118 of the housing 111, respectively. The first commercial dispenser 110 is manufactured and sold by S. C. Johnson under the Glade® brand (the commercial dispenser 110 will hereinafter be referred to as the “Glade dispenser”). Although the refill 50 is described as being insertable into the Glade dispenser 110 of FIG. 3, the refill 50 may also be inserted into multiple other dispensers manufactured and sold by S. C. Johnson and having similar features for retaining the refill 50 therein.

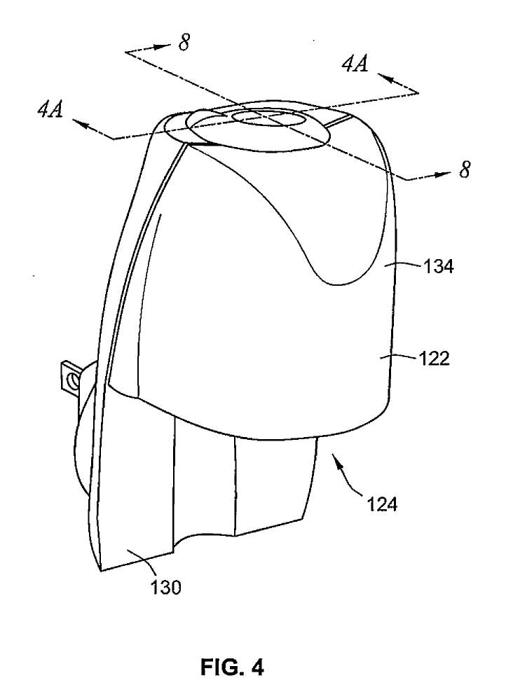

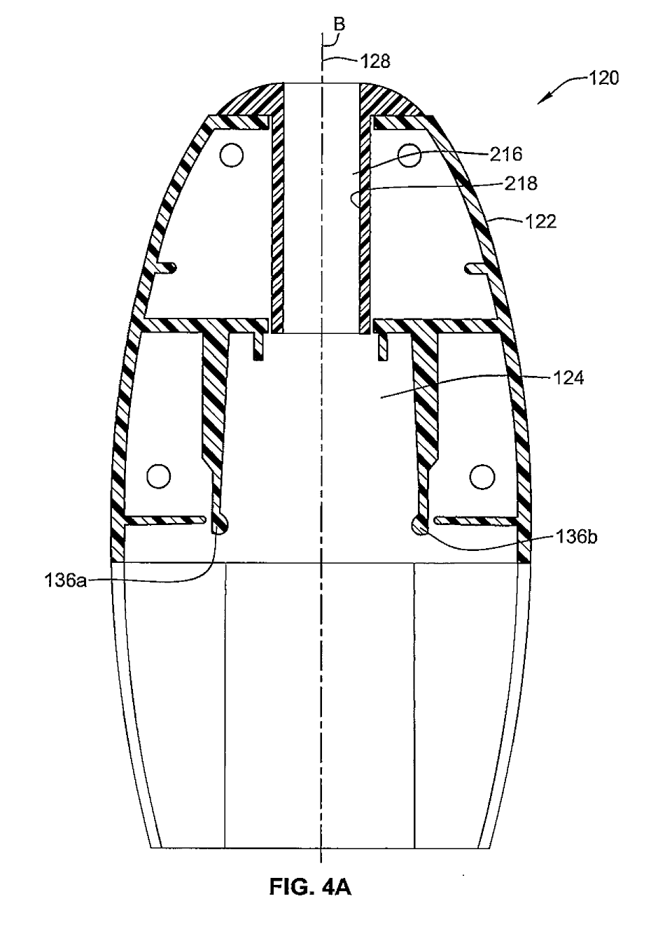

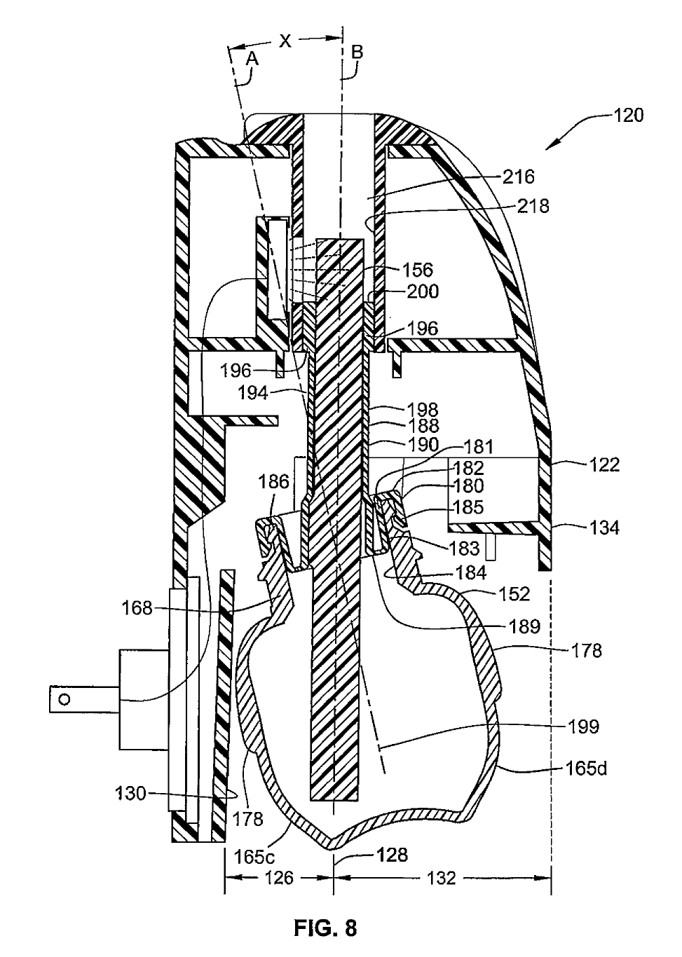

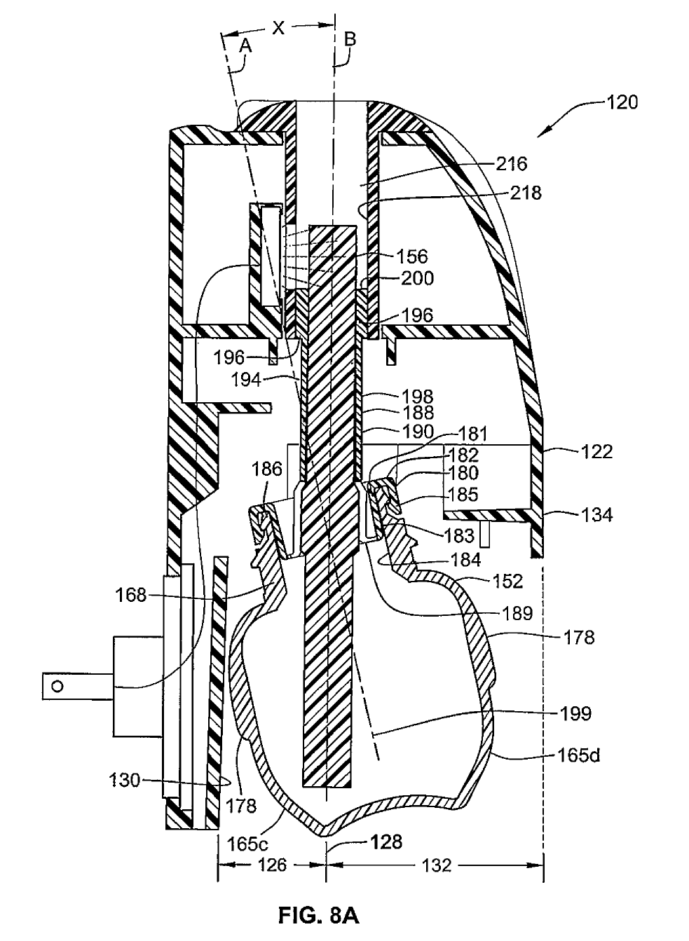

A second commercial dispenser 120, as seen in FIGS. 4 and 4A, is sold by Reckitt under the Air Wick® brand (the commercial dispenser 120 will hereinafter be referred to as the “Air Wick dispenser”). As discussed in the background herein, the disadvantage of the refill 50 is that it is not suitably adapted to be used in dispensers sold by companies other than S. C. Johnson, such as the Air Wick dispenser 120. In particular, the Air Wick dispenser 120 includes a housing 122 having a cavity 124 for accepting a refill. The housing 122 is dimensioned to generally accept a refill that is asymmetrical. More specifically, referring to FIG. 8, a first distance 126 between a centerpoint 128 of the cavity 124 and a rear wall 130 of the housing 122 is less than a second distance 132 between the centerpoint 128 and a front wall 134 of the housing 122. The refill (not shown) adapted to fit within the Air Wick dispenser 120 therefore includes similar dimensions such that the refill can be easily inserted into and accommodated within the Air Wick dispenser 120. Referring to FIG. 4A, the refill for use with the Air Wick dispenser 120 includes a collar extending outwardly from a neck thereof, wherein resilient latches 136a, 136b extending downwardly within the cavity 124 of the dispenser 120 grasp the collar to retain the refill in the dispenser, as discussed in greater detail hereinafter.

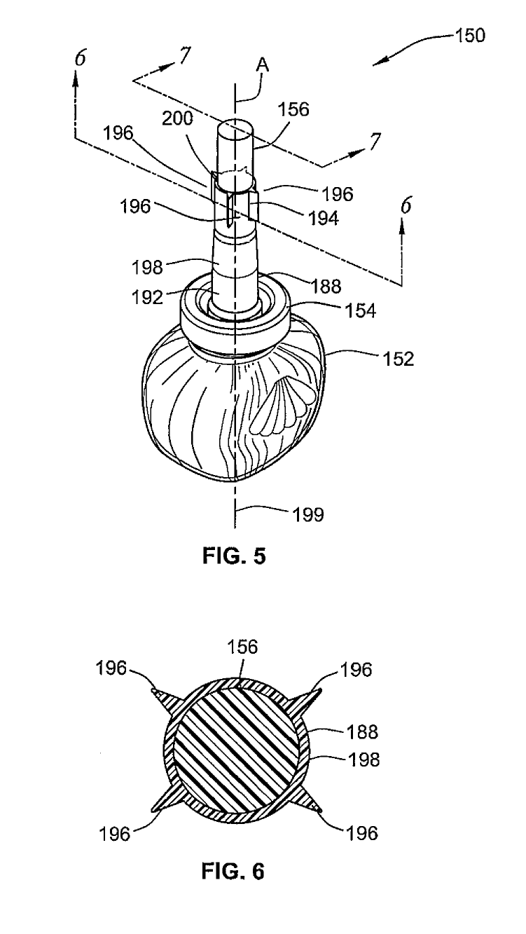

A first embodiment of a refill 150 adapted for use with at least the Glade dispenser 110 and the Air Wick dispenser 120 is depicted generally in FIGS. 5, 6, 7, and 8. Referring to FIGS. 5 and 6, the refill 150 includes a container 152, a plug assembly 154, a wick 156, and an optional cap (not shown). The container 152, the wick 156, and the optional cap (not shown) are substantially identical to the container 52, the wick 56, and the cap 58 of FIG. 1, but the plug assembly 154 is different. The plug assembly 154 includes an outer annular ring 180 and an inner annular ring 181 that are generally vertical and connected by a generally horizontal surface 182. The inner annular ring 181 is designed to have an outer surface 183 that is complementary to and abuts an inner surface 184 forming an opening 174 of the container 152. The outer annular ring 180 includes an inwardly extending annular projection 185 for snapping onto an outwardly extending projection 186 on a neck 168 of the container 152. The plug assembly 154 further includes a sheath 188 having an inwardly extending annular portion 189 and an upwardly extending cylindrical portion 190. The annular portion 189 and the cylindrical portion 190 surround at least a portion of the wick 156. Although one particular mechanism for retaining the plug assembly 154 within the refill 150 is utilized, any known mechanism for retaining such plug assembly 154 may be utilized, including but not limited to, adhesive, an interference fit, a snap fit, and the like.

Still referring to FIGS. 5 and 6, surface projections in the form of ribs 196 are disposed on an outer surface 198 of the sheath 188. The ribs 196 may be integrally molded with the sheath 188, may be applied to the sheath 188 during the manufacturing process, for example using an adhesive, or may be applied to the sheath 188 by the consumer. The ribs 196 are generally parallel to an axial extent 199 of the sheath 188 and are spaced about an equal distance from each other around the entire circumference of the outer surface 198. The ribs 196 generally extend along the upper portion 194 of the sheath 188 from a top edge 200 of the sheath 188 downwardly toward the lower portion 192 of the sheath 188. The ribs 196 extend radially outwardly from the outer surface 198 of the sheath 188 between about 0.25 mm and about 5, more preferably between about 0.75 mm and about 2 mm, and most preferably about 1.5 mm. Although the ribs 196 are shown as elongate protrusions having a triangular cross-section extending along a length of the sheath 188, the ribs 196 may alternatively or in addition comprise other surface irregularities or protrusions of other cross-section or size that extend outwardly from the sheath 188 in the form of projections. As will be discussed in greater detail hereinafter, the ribs 196 or other protrusions are adapted to be disposed within a channel in one or more dispensers. The size and shape of the ribs 196 or other protrusions are optimized to retain the refill 150 within one or more dispensers while still allowing air flow through the channel in which the sheath 188 and ribs 196 or protrusions are disposed. Although four ribs 196 are depicted in FIGS. 5 and 6, any number size, or orientation of ribs 196 may be utilized as long as the functions of the ribs 196, the sheath 188, and the refill 150 are not compromised, as discussed in greater detail hereinafter. Further, although the ribs 196 are depicted as being disposed along the upper portion 194 of the sheath 188, the ribs 196 may be disposed around the lower portion 192 of the sheath 188 or at a point somewhere between the upper portion 194 and the lower portion 192 of the sheath 188 (not shown).

In a further embodiment, the ribs are circumferential around the sheath 88 and gaps are formed therebetween to allow air to flow through a device in which the refill is disposed. The only requirements are that the ribs form an interference fit between the sheath/wick and walls forming the channel of the device and that air flow is not completely disrupted through the channel.

At least a portion of the sheath 188 is preferably made of a flexible material. In particular, in the embodiment of FIGS. 5 and 6, the inwardly extending annular portion 189 and optionally a portion of the upwardly extending cylindrical portion 190 are made of a flexible material, such that the sheath 188 may pivot about the annular portion 189. Optionally, the entire sheath 188 and/or plug assembly 154 may be made of a flexible material. Allowing the wick 156 and the sheath 188 to pivot about the annular portion 189 allows the wick 156 (and the sheath 188) to be disposed in an upright position, as seen in FIG. 5, or at an angle with respect to an axis A of the refill 150, as shown in FIG. 8. In particular, the wick 156 (and the sheath 188) may be pivoted to an angle of up to about 60 degrees from the axis A of the refill 150. The sheath 188 may be made out of polyethylene including low density polyethylene, high density polyethylene, and the like. Other suitable materials include polypropylene and other co-polymers, such as, for example Barex® resins. Any of the materials used to construct the sheath 188 should preferably be compatible with the volatilizing liquid.

Alternatively, the sheath 188 and/or plug assembly 154 may be made out of a non-flexible material and the wick 156 pivoting may be accomplished in other ways. For example, material may be removed from portions of the sheath 188 to form slots that create hinges or pivot points to facilitate angular movement of the wick 156. As shown in FIG. 8A, portions of material are removed from the sheath 188 around a circumference thereof to create one or more interference points. Preferably, there are two interference points spaced to allow the wick 156 to be retained by the sheath 188, while at the same time to allow the sheath 188 with the wick 156 disposed therein to pivot as described previously herein.

The refill 150 may be inserted into and used with both the Glade and Air Wick dispensers 110, 120. In order to use the refill 150 of FIGS. 5 and 6, if a cap (not shown) is utilized, the cap must first be removed. Referring to FIG. 7, when a user desires to utilize the refill 150 with the Glade dispenser 110, the user inserts the wick 156 and sheath 188 of the refill 150 into a channel 210 formed within the dispenser 110. As the refill 150 is inserted into the dispenser 110, the wick 156 and the sheath 188 are upright such that axes of the wick 156 and the sheath 188 are coincident with the axis A of the refill 150. As described in detail above, raised portions 178 on the container 152 are retained within the groove 112 and the aperture 116 in the housing 111 of the Glade dispenser 110. In addition, the ribs 196 abut a cylindrical wall 212 forming the channel 210 within the housing 111 to provide an additional mechanism by which to retain the refill 150 within the Glade dispenser 110. Once the refill 150 is secured in the Glade dispenser 110, at least a portion of the wick 156 is preferably exposed to allow heat from a heater (not shown) in the dispenser 110 to evaporate a volatile material 162 that is moved to a top portion of the wick 156 through capillary action

Referring to FIG. 8, when the user desires to utilize the refill 150 in the Air Wick dispenser 120, the user must first angle the refill 150 with respect to the housing 122 due to the unique dimensions of the housing 122, as discussed in detail above. Thereafter, the wick 156 and the sheath 188 of the refill 150 are inserted into the cavity 124 formed by the housing 122 of the dispenser 120. As the wick 156 and the sheath 188 are pushed into the cavity 124, the sheath 188 pivots about the annular portion 189 such that axes of the wick 156 and sheath 188 are no longer coincident with the axis A, but instead, are angled toward the an opposing wall 165c, 165d disposed adjacent the rear wall 130 of the housing 122 of the dispenser 120. The wick 156 and sheath 188 are preferably disposed at an angle X with respect to the axis A. The angle X is preferably between about 5 and about 45 degrees with respect to the axis A, more preferably between about 10 and about 30 degrees, and most preferably about 20 degrees. As the wick 156 and sheath 188 pivot, the wick 156 and sheath 188 are no longer centered within the container 152 between the third and fourth opposing walls 165c, 165d, but instead, the wick 156 and sheath 188 are offset toward the opposing wall 165c, 165d adjacent the rear wall 130 of the dispenser 120. Flexing of the sheath 188 about the annular portion 189 consequently tilts the container 152 with respect to an axis B of the dispenser 120 at an angle similar to the angle X. Still referring to FIG. 8, once the refill 150 is secured in the Air Wick dispenser 120, the wick 156 and sheath 188 are disposed within a channel 216 within the cavity 124 and remain aligned with the axis B of the dispenser 120. Further, the ribs 196 abut a cylindrical wall 218 forming the channel 216 to retain the refill 150 within the dispenser 120 without the refill 150 engaging or being retained by the latches 136a, 136b in the Air Wick dispenser 120. Once the refill 150 is secured in the dispenser 120, at least a portion of the wick 156 is preferably exposed to allow heat from a heater (not shown) in the dispenser 120 to evaporate volatile material 162 that is moved to a top portion of the wick 156 through capillary action. As noted above, the number, size, and orientation of the ribs 196 may be varied as long as the ribs 196 still retain the refill 150 within the dispenser 120 while still allowing air flow through the channel 216.

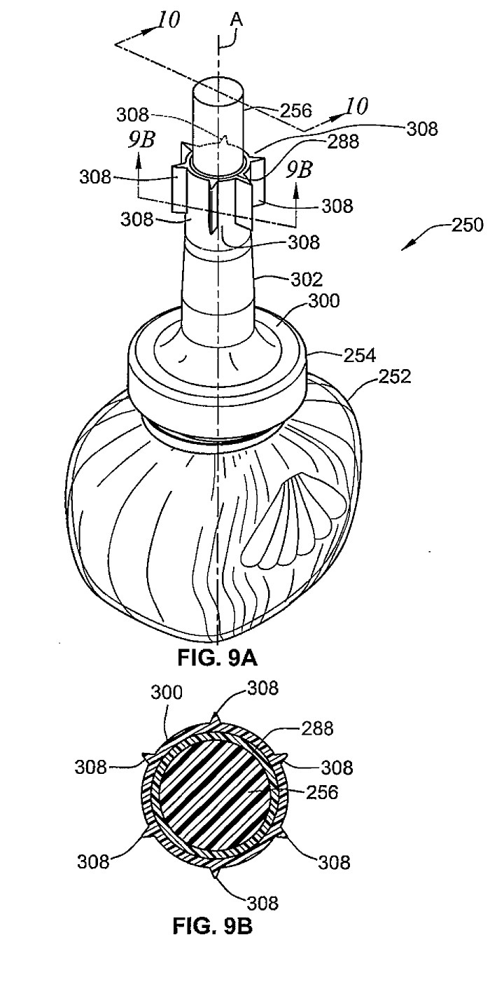

A second embodiment of a refill 250 adapted for use with at least the Glade dispenser 110 and the Air Wick dispenser 120 is shown generally in FIGS. 9A, 9B, 10, and 11. Referring to FIGS. 9A and 9B, the refill 250 includes a container 252, a plug assembly 254, a wick 256, and an optional cap (not shown). The container 252, the wick 256, and the cap are substantially identical to the container 52, the wick 56, and the cap 58 of FIG. 1. As with the embodiment of FIG. 5, the plug assembly 254 is different and includes an outer annular ring 280 and an inner annular ring 281 that are generally vertical and connected by a generally horizontal surface 282. The inner annular ring 281 is designed to have an outer surface 283 that is complementary to and fits against an inner surface 284 forming an opening 274 of the container 252. The outer annular ring 280 includes an inwardly extending annular projection 285 for snapping onto an outwardly extending projection 286 on a neck 268 of the container 252. The plug assembly 254 further includes a sheath 288 having an inwardly extending annular portion 289 and an upwardly extending cylindrical portion 290. The annular portion 289 and the cylindrical portion 290 surround and retain the wick 256 in the same manner as discussed above with respect to FIGS. 1-3. Similar to the embodiment of FIGS. 5-8, the inwardly extending annular portion 289 and optionally a portion of the upwardly extending cylindrical portion 290 are made of a flexible material, such that the sheath 288 may pivot about the annular portion 289 to rotate the wick 256 into different angular positions, as discussed in detail relative to the embodiment of FIGS. 5-8. Optionally, the entire sheath 288 may be made of a flexible material.

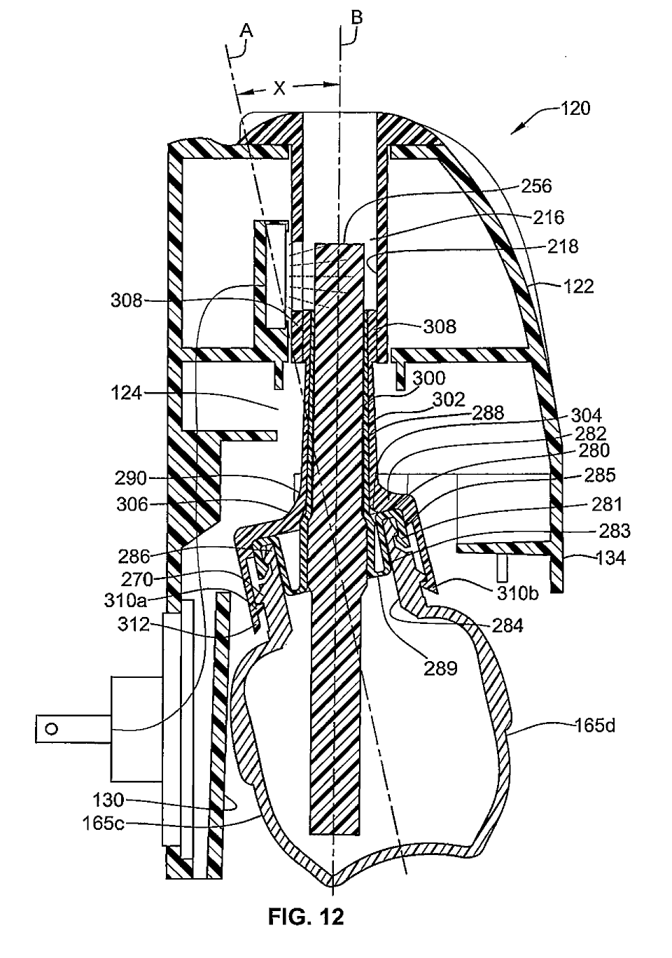

Referring to FIG. 9A, the refill 250 is provided, either already attached to or in a package with, an adapter 300 that is preferably complementary in shape to and covers the sheath 288 thereof. The adapter 300 includes an upwardly extending hollow cylindrical portion 302 connected to an annular flared base 304 by a flexible portion 306. At least the flexible portion 306 of the adapter 300 is made of a flexible material and may be made of the same or different material as the flexible portion(s) of the sheath 288. Suitable examples of flexible material include, but are not limited to, low density polyethylene, high density polyethylene, and/or mixtures thereof. Alternatively, the flexible portion 306 (and other portions or all of the adapter 300) may be formed to create a flexible area (e.g., thicker and thinner portions of material may form flexible pleats). The cylindrical portion 302 further includes ribs 308 extending outwardly therefrom similar to the ribs 196 described with respect to the embodiment of FIGS. 5-8. The number, size, and orientation of the ribs 308 may be varied as long as the ribs 308 retain the refill 250 within the dispenser 120 while still allowing air flow through the channel 216. The adapter 300 slides over a top of the refill 250 and substantially surrounds the sheath 288 and the plug assembly 254 while allowing the wick 256 to extend upwardly out of the sheath 288 and the adapter 300, as shown in FIG. 9A. As the adapter 300 is inserted onto the sheath 288, one or more inwardly extending annular latches 310a, 310b on the adapter 300 snap over the plug assembly 254. Alternatively, as shown in a third embodiment of FIG. 12, the latches 310a, 310b on the adapter 300 may interact directly with a threaded portion 270 on the neck 268 of the container 252, for example, through a snap fit or a rotation about the threaded portion 270. Still optionally, the adapter 300 may be attached to the plug assembly 254 or the container 252 in any other manner known in the art. The adapter 300 also preferably includes an annular ledge 312 that protrudes outwardly a distance greater than the neck 268.

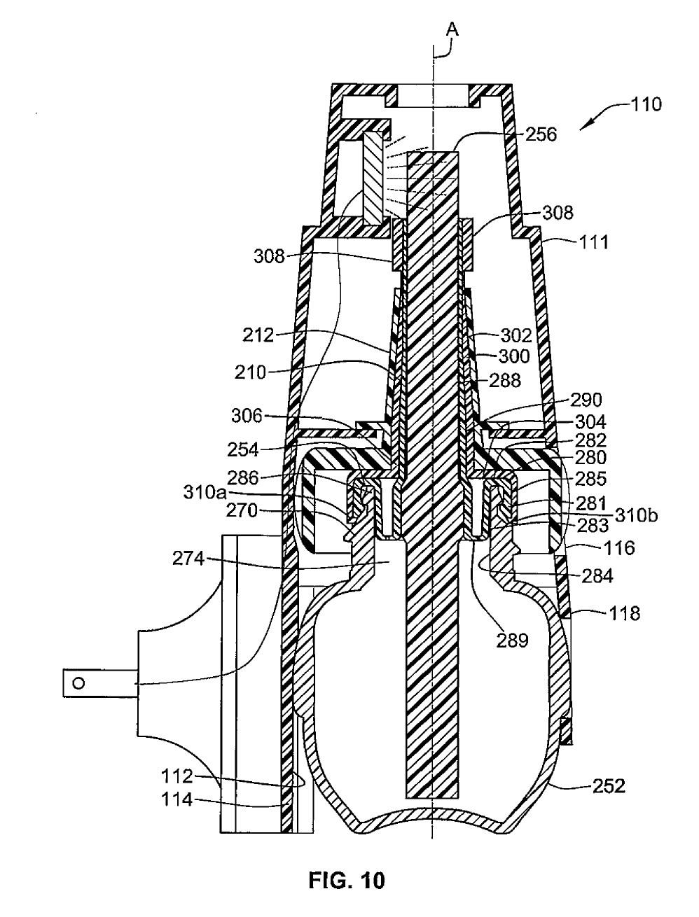

The refill 250 may be inserted into and used with both the Glade and Air Wick dispensers 110, 120. Preferably, the refill 250 is sold with the adapter 300 in the same package. In such case, the refill 250 is inserted (without the adapter 300 attached thereto) into the Glade dispenser 110, as shown in FIG. 10 and retained in the same manner as in FIG. 7 (by way of raised portions 178 and the ribs 308). The adapter 300 may also be sold separately from the refill 250 or the refill 250 may be sold with the adapter 300 attached thereto, wherein the adapter 300 is preferably (although not necessarily) removed for use with the Glade dispenser 110.

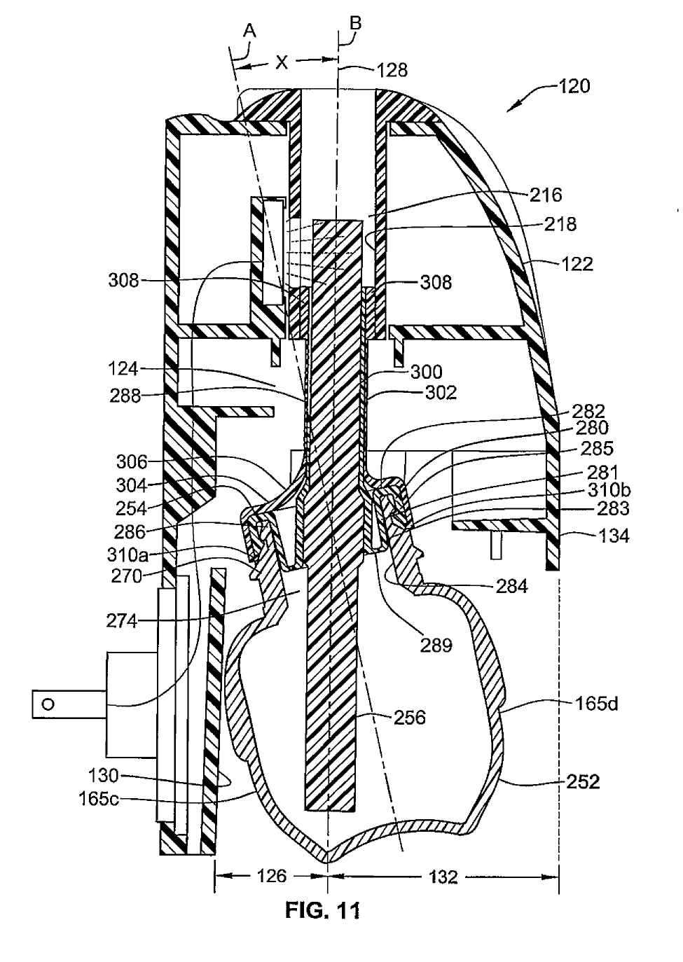

As depicted in FIG. 11, before the refill 250 is inserted into the Air Wick dispenser 120, the adapter 300 is attached to the refill 250 in the manner described above, if the refill 250 is not provided with the adapter 300 affixed thereto. The refill 250 with adapter 300 attached thereto is thereafter inserted into the dispenser 120 by angling the refill 250 as discussed with respect to FIG. 8. As the wick 256 and the sheath 288 are pushed into the cavity 124 of the housing 122 of the dispenser 120, the sheath 288 and the adapter 300 pivot about the annular portion 289 and the flexible portion 306, respectively, such that the wick 256, the sheath 288, and the adapter 300 are no longer coincident with the axis A of the refill 250, but instead, are angled toward an external surface 265c, 265d of the container 252 disposed adjacent the rear wall 130 of the housing 122. Upon insertion, the wick 265, the sheath 288, and the adapter 300 are disposed at the angle X degrees with respect to the axis A, wherein the angle X is discussed in greater detail above with respect to FIGS. 5-8. As pivoting occurs, the wick 256, the sheath 288, and the adapter 300 are no longer centered within the container 252 between third and fourth opposing walls 265c, 265d, but instead, the wick 256 and sheath 288 are angled toward the wall 265c, 265d adjacent the rear wall 130 of the housing 122. Flexing of the sheath 288 and the adapter 300 about the annular portion 289 and the flexible portion 306, respectively, consequently tilts the container 252 with respect to the axis B of the dispenser 120, as discussed in detail above with respect to the embodiment of FIGS. 5-8. Still referring to FIG. 11, once the refill 250 is secured in the Air Wick dispenser 120, the wick 256, the sheath 288, and the adapter 300 are disposed within the channel 216 and remain aligned with the axis B of the dispenser 120. Further, the ribs 308 abut the cylindrical wall 218 forming the channel 216 to retain the refill 250 within the dispenser 120 without the refill 250 engaging or being retained by the latches 136a, 136b in the Air Wick dispenser 120. Once the refill 250 is secured in the dispenser 120, at least a portion of the wick 256 is preferably exposed to allow heat from a heater (not shown) in the dispenser 120 to evaporate volatile material 262 that is moved to the top portion of the wick 256 through capillary action.

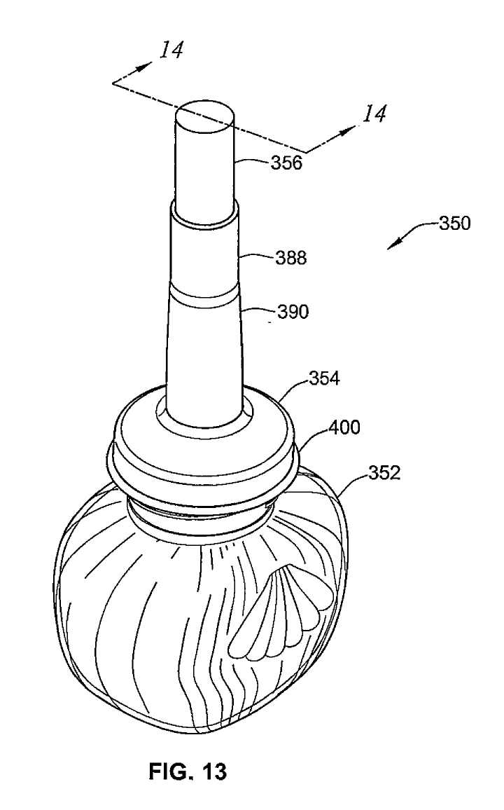

In a fourth embodiment, as depicted in FIGS. 13, 14, 15A, and 15B, a refill 350 adapted for use with at least the Glade dispenser 110 and the Air Wick dispenser 120 includes a container 352, a plug assembly 354, a wick 356, and an optional cap (not shown). The container 352, the wick 356, and the cap are identical to the container 52, the wick 54 and the cap 58 of FIG. 1, while the plug assembly 354 is different. As with the embodiment of FIG. 5, the plug assembly 354 includes an outer annular ring 380 and an inner annular ring 381 that are generally vertical and connected by a generally horizontal surface 382. The inner annular ring 381 is designed to have an outer surface 383 that is complementary to and fits against an inner surface 384 forming an opening 374 of the container 352. The outer annular ring 380 includes an inwardly extending annular projection 385 for snapping onto an outwardly extending projection 386 on a neck 368 of the container 352. The plug assembly 354 further includes a sheath 388 having an inwardly extending annular portion 379 and an upwardly extending cylindrical portion 380. The annular portion 389 and the cylindrical portion 390 surround and retain the wick 356 in a manner similar to previous embodiments 1. Unlike the previous embodiments, the outer annular ring 380 includes an outwardly extending annular lip 400. Although the lip 400 is depicted as continuous, the lip 400 may alternatively be formed of multiple discrete lip portions (not shown). Still optionally, the annular lip 400 may be integral with or an attachment for the plug assembly 354 or may be integral with or an attachment for a portion of the refill 350, for example, the neck 368, as discussed in greater detail hereinbelow.

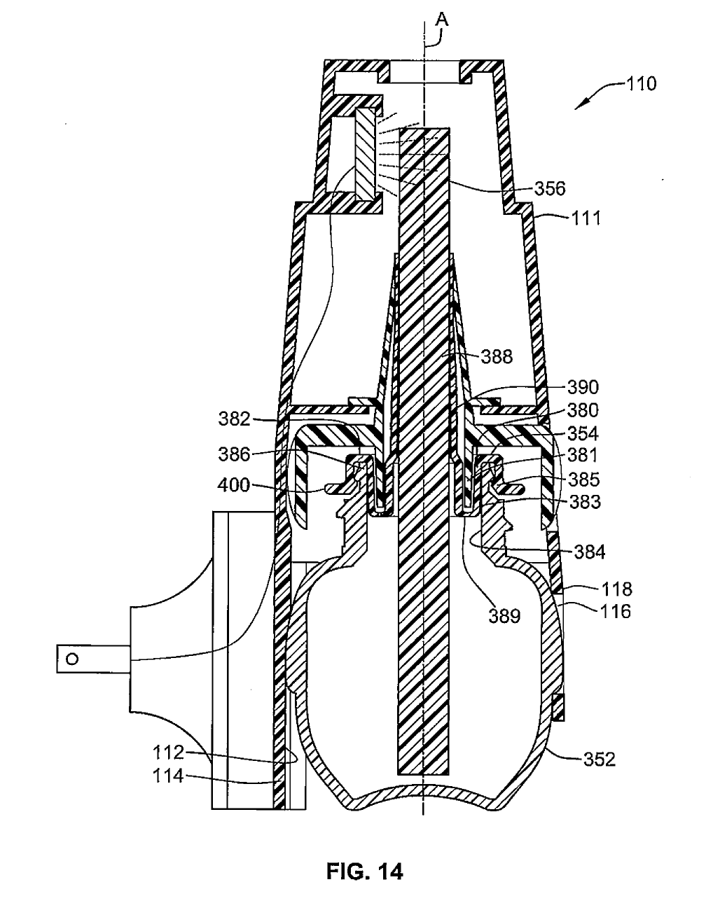

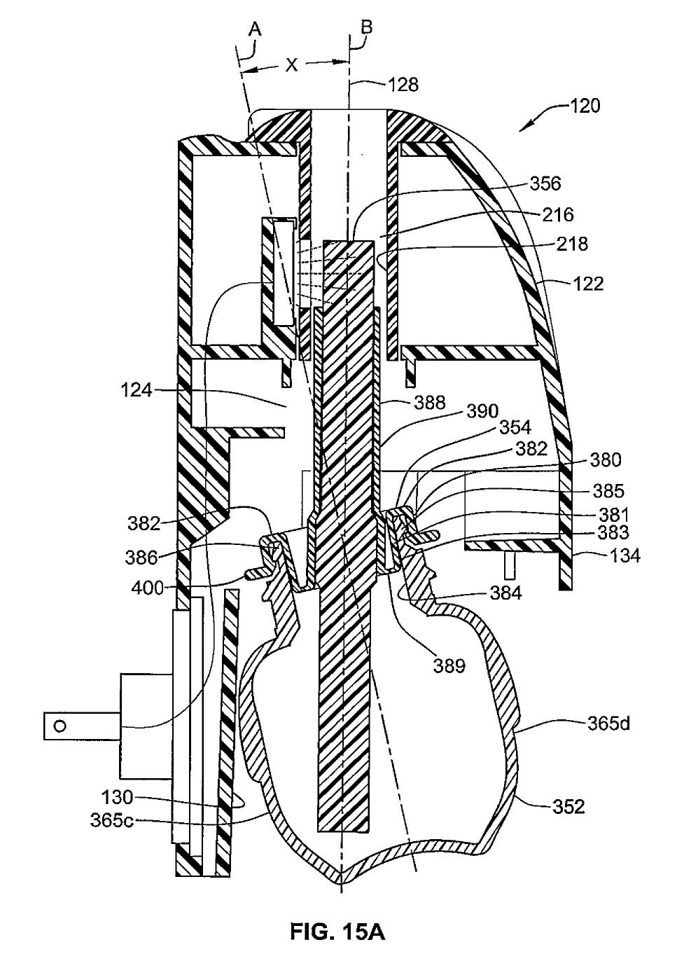

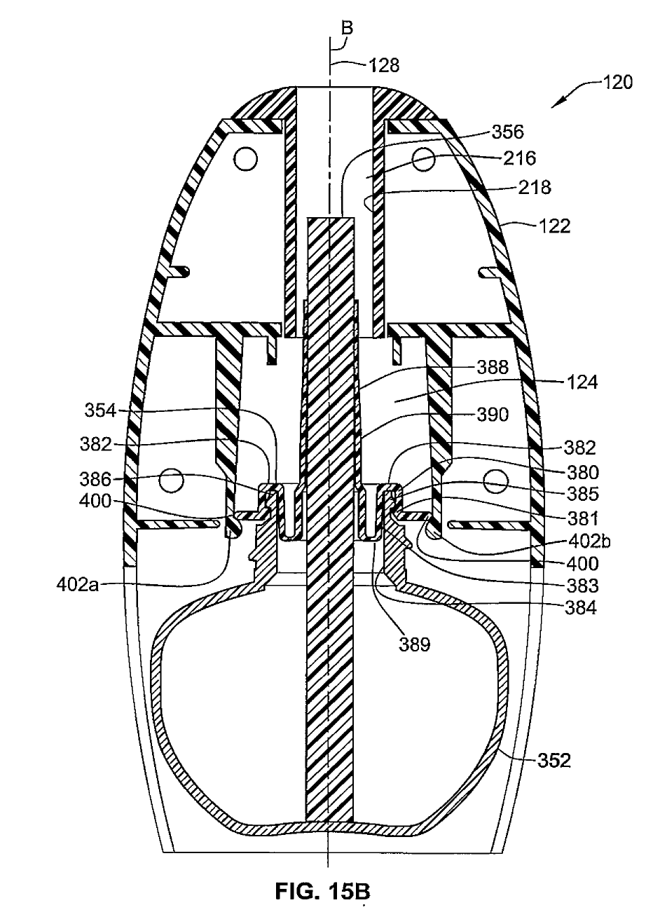

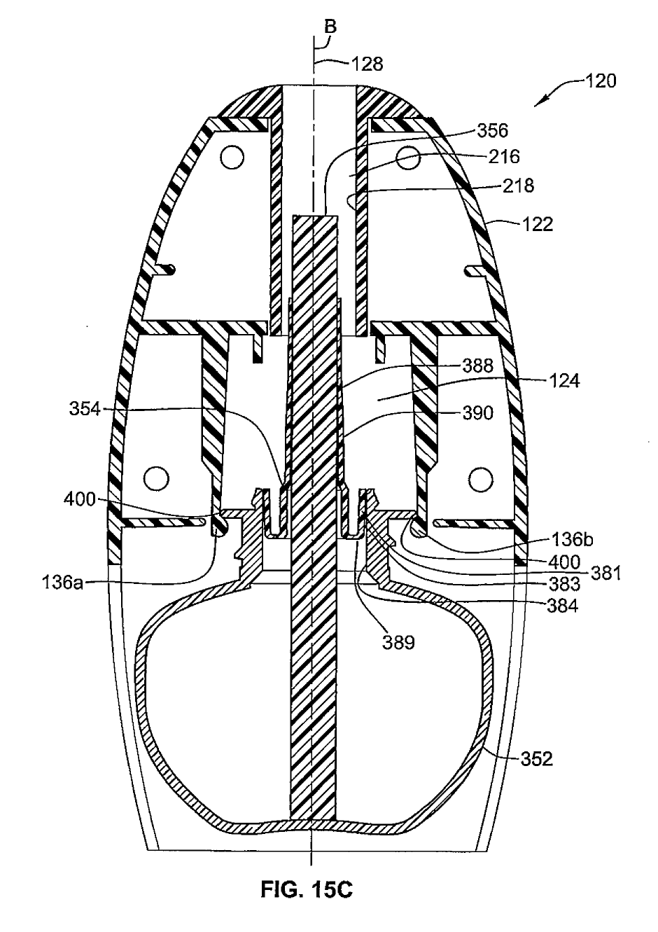

The refill 350 may be inserted into and used with both of the Glade and Air Wick dispensers 110, 120. Specifically, the refill 350 may be inserted into the Glade dispenser 110, as shown in FIG. 14, in the same manner as described with respect to FIGS. 1-3. In order to insert the refill 350 into the Air Wick dispenser 120, the refill 350 must be angled as seen in FIG. 15A with respect to the dispenser 120, as discussed in detail with respect to the embodiment of FIGS. 5-8. As the wick 356 and the sheath 388 are pushed into the cavity 124 of the housing 122 of the dispenser 120, the sheath 388 pivots about the annular portion 389 such that the wick 356 and the sheath 388 are no longer coincident with an axis A of the refill 350, as seen in FIG. 15A. Instead, the wick 356 and the sheath 388 are angled toward a wall 365c, 365d of the container 352 disposed adjacent the rear wall 130 of the housing 122 of the dispenser 120. The wick 356 and the sheath 388 are angled in a manner as discussed above with respect to FIGS. 8 and 11.

Referring to FIG. 15B, as the refill 350 is inserted into the dispenser 120, opposing flexible and resilient latches 136a, 136h disposed within the cavity 124 of the dispenser 120 move outwardly to accommodate the annular lip 400. As the annular lip 400 passes the latches 136a, 136b, the latches 136a, 136b return to their original position, wherein the latches 136a, 136b interfere with the lip 400 to prevent removal of the refill 350 from the dispenser 120. The refill 350 may be removed from the dispenser 120 by exerting a downward force on the refill 350, which, in turn, exerts downward and outward pressure from the lip 400 on the latches 136a, 136b, thereby forcing the latches 136a, 136b outwardly such that the refill 350 may be removed therefrom.

In a variation of the embodiment of FIGS. 13-15B, and as seen in FIG. 15C, the plug assembly 354 may only include the inner annular ring 381, the annular portion 389 extending inwardly therefrom, and the cylindrical portion 390 surrounding and retaining the wick 356. The outer surface 383 of the inner annular ring 381 may be held in engagement with the inner surface forming the opening 374 by a friction fit, adhesive, or any other means known in the art. The annular lip 400 is integral with (or attached to) the neck 368 of the refill 350, such that the annular lip 400 may be retained by the latches 136a, 136b, as discussed in detail with respect to FIG. 15B.

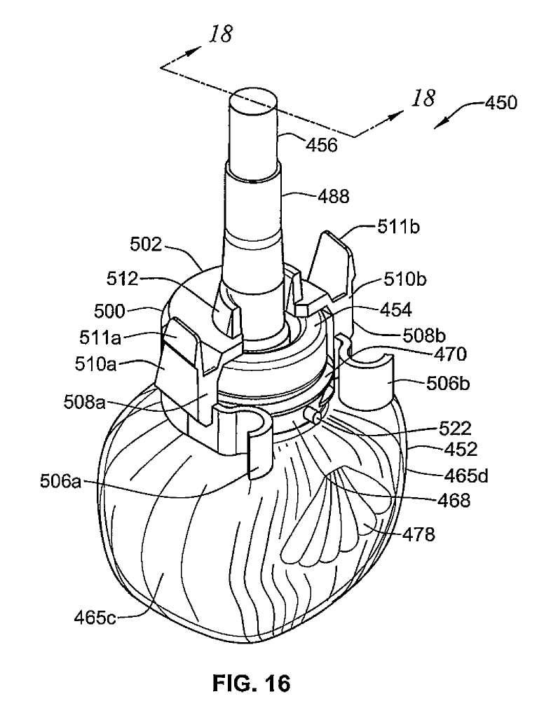

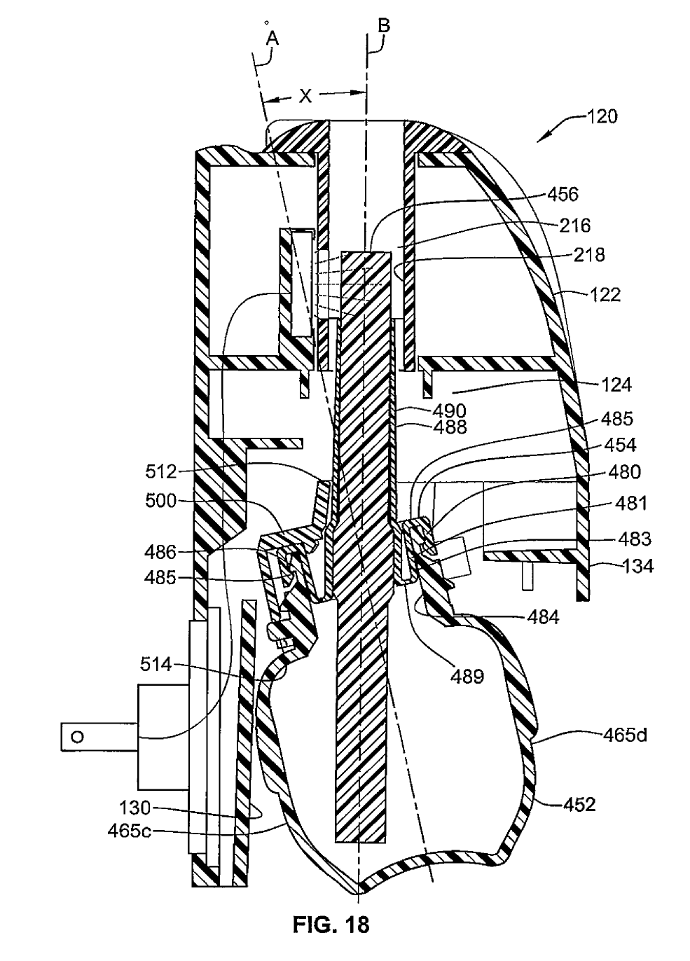

Turning now to FIGS. 16, 17A, 17B, 18 and 19, a fifth embodiment of a refill 450 for use with at least the Glade and Air Wick dispensers 110, 120 includes a container 452, a plug assembly 454, and a wick 456. The container 452 and the wick 456 are substantially identical to the container 52 and the wick 56 of FIG. 1. In addition, the plug assembly 454 is substantially similar to the plug assemblies 154, 254 (without ribs) discussed hereinabove with respect to the embodiments of FIGS. 5-8 and 9A, 9B, 10, and 11, respectively. In particular, the plug assembly 454 includes an outer annular ring 480 and an inner annular ring 481 that are generally vertical and connected by a generally horizontal surface 482. The inner annular ring 481 is designed to have an outer surface 483 that is complementary to and fits against an inner surface 484 forming an opening 474 of the container 452. The outer annular ring 480 includes an inwardly extending annular projection 485 for snapping onto an outwardly extending projection 486 on a neck 468 of the container 452. The plug assembly 454 further includes a sheath 488 having an inwardly extending annular portion 489 and an upwardly extending cylindrical portion 490. The annular portion 489 and the cylindrical portion 490 surround and retain the wick 456 as discussed in detail hereinabove.

At least a portion of the sheath 488 is preferably made of a flexible material. In particular, the annular portion 489 and optionally a portion of the cylindrical portion 490 are made of a flexible material, such that the sheath 488 may pivot about the annular portion 189, in a manner similar to that described with respect to the embodiment of FIGS. 5-8. Optionally, the entire sheath 488 may be made of a flexible material.

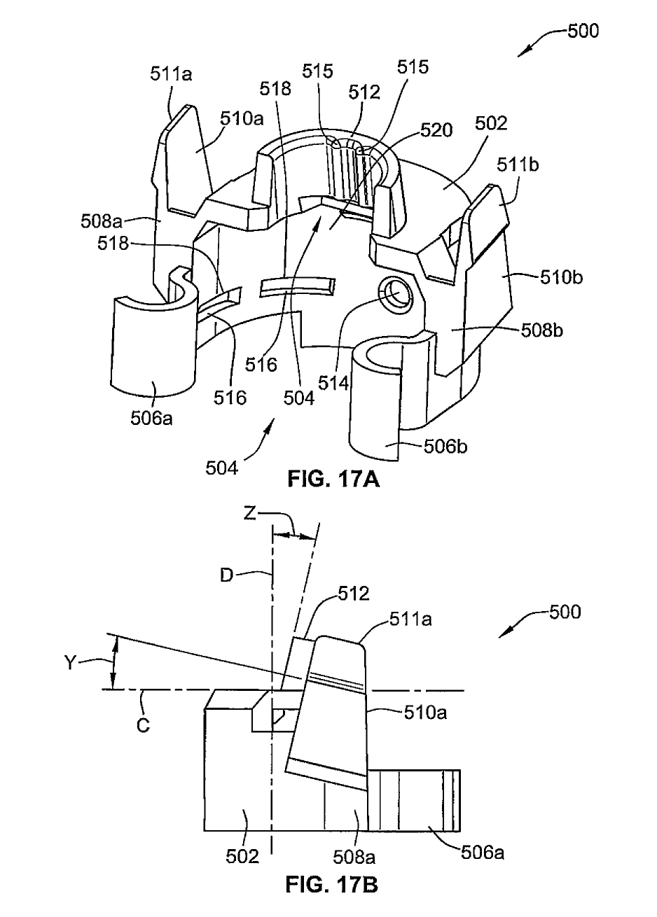

As best seen in FIGS. 17A and 17B, a substantially semi-circular attachment 500 in the form of a partial ring 502 with a central void 504 is further included with the refill 450 and may be attached to the refill 450 for insertion into one or more dispensers. Although the attachment 500 herein is disclosed as being semi-circular in shape, any other shaped attachments are possible, as long as the attachment retains the same functions as described herein. The refill 450 may be provided with the attachment 500 affixed thereto, in a package with the attachment 500, or completely separate from the attachment 500. The ring 502 includes opposing resilient outwardly facing C-shaped clasps 506a, 506b located at ends 508a, 508b of the partial ring 502. The partial ring 502 further includes upwardly extending latches 510a, 510b disposed on opposing sides of the void 504 adjacent the clasps 506a, 506b. The latches 510a 510b include outwardly extending projections 511a, 511b, the function of which will be discussed in greater detail hereinafter. The projections 511a, 511b are preferably angled downwardly from a third wall 465c to a fourth wall 465d of the container 452 at an angle Y with respect to a horizontal axis C that extends through and defines an axial extent of a top wall 513 of the attachment 500 that is preferably between about 5 degrees and about 45 degrees, more preferably between about 10 degrees and about 30 degrees, and most preferably about 20 degrees. A semi-circular neck portion 512 extends upwardly from the partial ring 502, wherein the neck portion 512 is disposed at an angle Z with respect to an axis D of the attachment 500. The angle Z is preferably between about 5 degrees and about 45 degrees, more preferably between about 10 degrees and about 30 degrees, and most preferably about 20 degrees. The neck portion 512 may optionally be omitted such that the wick 456 is retained in alignment with the axis D (in a vertical position). In such case, if the wick 456 needs to be pivoted for use within one or more dispensers, the pivotable wick 456 may be pivoted by the user upon insertion, as discussed in greater detail hereinabove. Still further, the neck portion 512 may further include one or more projections in the form of teeth 515 (FIG. 17A), disposed on an inner surface 520 thereof. The teeth 515 are adapted to assist in grasping and retaining the sheath 488 in place once the attachment 500 is inserted onto the refill 450. The partial ring 502 optionally includes one or more positioning apertures 514 disposed therethrough.

Referring to FIG. 17A, at least one indentation 516 forming at least one shelf 518 is disposed in the inner surface 520 of the ring 502 to aid in retaining the attachment 500 on the container 452. If more than one shelf 518 is present, each shelf 518 may be vertically staggered to correspond to threaded portions 470 of the neck 468. Optionally, other projections (not shown) or indentations may be disposed on the neck 468 of the refill 450 for cooperation with the at least one shelf 518 or at least one projection to aid in retaining the attachment 500 thereto. Preferably, the attachment 500 is made of a more rigid material than the wick 456 and the sheath 488. For example, suitable materials may include polypropylene, polyethylene, high density polyethylene, low density polyethylene, and the like. Any material chosen should be compatible with the chemical composition of the volatile material.

The refill 450 may be inserted into and used with both the Glade and Air Wick dispensers 110, 120. Preferably, the refill 450 is sold in the same package with the attachment 500, but the attachment 500 is not secured to the refill 450. In such case, the attachment 500 is not necessary for use with the Glade dispenser 110 and thus, does not need to be secured to the refill 450. The refill 450 is therefore inserted into the Glade dispenser 110 in the same manner as discussed above with respect to FIGS. 1-3.

When the refill 450 is used with the Air Wick dispenser 120, the attachment 500 is secured to the neck 468 of the container 452. In particular, the attachment 500 may be slid horizontally around the neck 468. Pressure exerted on the attachment 500 is transferred to the neck 468, which is less resilient than the attachment 500, thereby causing the clasps 506a, 506b to flex outwardly until the clasps 506a, 506b pass the neck 468. Once past the neck 468, the clasps 506a, 506b return to their original position, wherein the attachment 500 is disposed around and retained on the neck 468. As the attachment 500 is positioned on the refill 450, the teeth 515 abut at least a portion of the sheath 488 to assist in retaining the attachment 500 on the refill 450 by way of the sheath 488. Alternatively, the attachment 461 can be slid downwardly over the wick 456 and the neck 468. As the attachment 500 is affixed to the neck 468 of the container 452, the angled neck portion 512 contacts the wick 456 and pivots the wick 456 and the sheath 488 about the annular portion 489 into a pre-set (by the attachment 500) angled position. The indentations 516 formed in the attachment 500 interact with the threaded portion 470 (or another feature on the refill 450) to assist in retaining the attachment 500 on the container 454 in a direction parallel to the wick 456. In addition, if utilized, the aperture 514 disposed through the attachment 500 is adapted to receive a projection 522 (FIG. 16) that extends outwardly from the neck 468 of the container 452 to help the user orient the attachment 500.

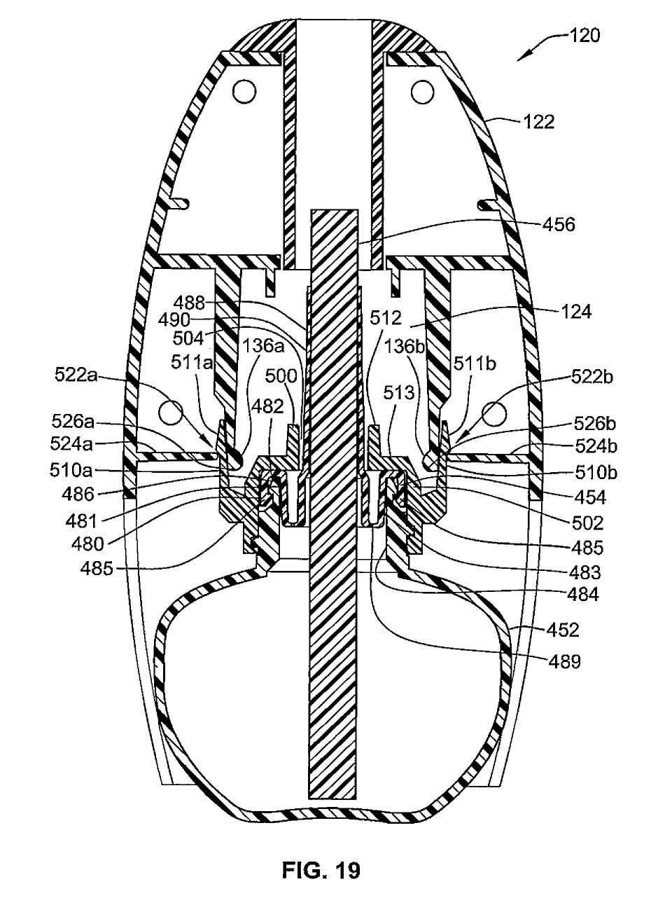

After the attachment 500 is secured to the refill 450, the refill 450 is inserted into the Air Wick dispenser 120 in the manner shown in FIG. 19. The refill 450 is inserted into the dispenser 120 by holding the refill 450 with the wick 456 and the sheath 488 angled toward the user. The wick 456 and the sheath 488 are thereafter inserted into the cavity 124 of the housing 122 of the dispenser 120, as discussed in greater detail above. The wick 456 and the sheath 488 are retained at the angle Z with respect to an axis A of the refill 450. As the refill 450 is inserted into the dispenser 120, the projections 511a, 511b extending outwardly from the latches 510a, 510b slide through gaps 522a, 522b formed between the resilient latches 136a, 136b disposed within the cavity 124 of the dispenser 122 and horizontal walls 524a, 524b aligned with bottom portions 526a, 526b of the latches 136a, 136b. In particular, the latches 510a, 510b flex inwardly to allow the projections 511a, 511b to pass through the gaps 522a, 522b. Once the projections 511a, 511b pass through the gaps 522a, 522b, the latches 510a, 510b flex outwardly to prevent downward movement of the projections 511a, 511b. The refill 450 with attachment 500 is retained in the Air Wick dispenser 120 without the refill 250 engaging or being retained by the latches 136a, 136b in the Air Wick dispenser 120. To remove the refill 450, a user must exert downward force, which forces the projections 511a, 511b downwardly against the walls 524a, 524b, thereby causing the latches 510a, 510b and projections 511a, 511b to move inwardly, allowing removal of the attachment 500. The attachment 500 may alternatively be sold affixed to the refill 450, in which case, the attachment 500 would be removed for use with the Glade dispenser 110.

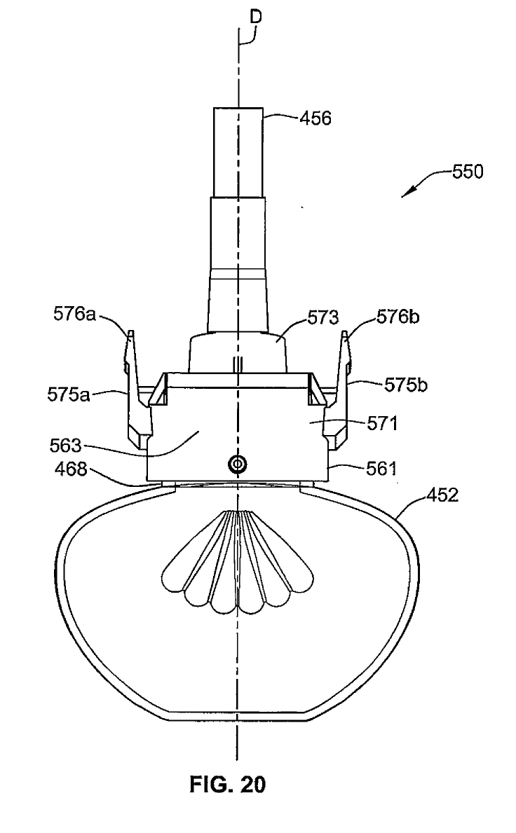

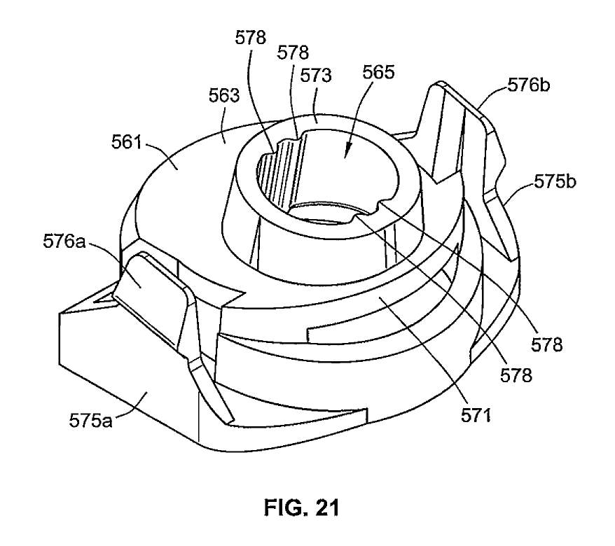

Referring now to FIGS. 20 and 21, a sixth embodiment of a refill 550 includes a container 452, a plug assembly 454, a wick 456, and a sheath 488 that are identical to those of the embodiment of FIGS. 16-19, and thus the reference numerals from such embodiment will be utilized for the present embodiment. An attachment 561 generally in the form of a ring 563 that includes a central channel 565 and may be packaged with or affixed to the refill 550 for sale thereof and may be used to attach the refill 550 to one or more dispensers, as discussed in greater detail hereinafter. The ring 563 includes a main body 571, an upwardly extending angled neck portion 573 and upwardly extending latches 575a, 575b disposed on opposing sides of the neck portion 573, wherein the latches 575a, 575b include outwardly extending projections 576a, 576b. The projections 576a, 576b are preferably angled downwardly from a third wall 465c to a fourth wall 465d of the container 452 at the angle Y, as discussed in detail with respect to the embodiment of FIGS. 16-19. An angle of the neck portion 573 is also similar to the angle Z of the neck portion 512 of the embodiment of FIGS. 16-19. The neck portion 573 may optionally be omitted such that the wick 456 is retained in alignment with an axis D of the attachment 561 (in a vertical position). In such case, if the wick 456 needs to be pivoted for use within one or more dispensers, the wick 456 may be pivoted by the user upon insertion, as discussed in greater detail hereinabove. Still further, the neck portion 573 may further include one or more projections in the form of teeth 578 disposed on an inner surface 580 thereof, wherein the teeth 578 perform the same function as the teeth 515 of FIG. 17A.

The refill 550 is used in the Glade and Air Wick dispensers 110, 120 in the same manner as disclosed with respect to the fifth embodiment of FIGS. 16-19, except that the attachment 561 (when sold unattached to the refill 550) is slid downwardly over the wick 456 and neck 468 for use with the Air Wick 120 dispenser or attached in any other manner known in the art, as seen in FIG. 19, until the attachment 561 snaps onto the plug assembly 454. Optionally, the attachment 561 may be affixed to the refill 550 in any other fashion known in the art. As with previous embodiments, the attachment 561 is not necessary (and not preferred) for use with the Glade dispenser 110. If sold attached to the refill 550, the attachment 561 may be removed for use with the Glade dispenser 110.

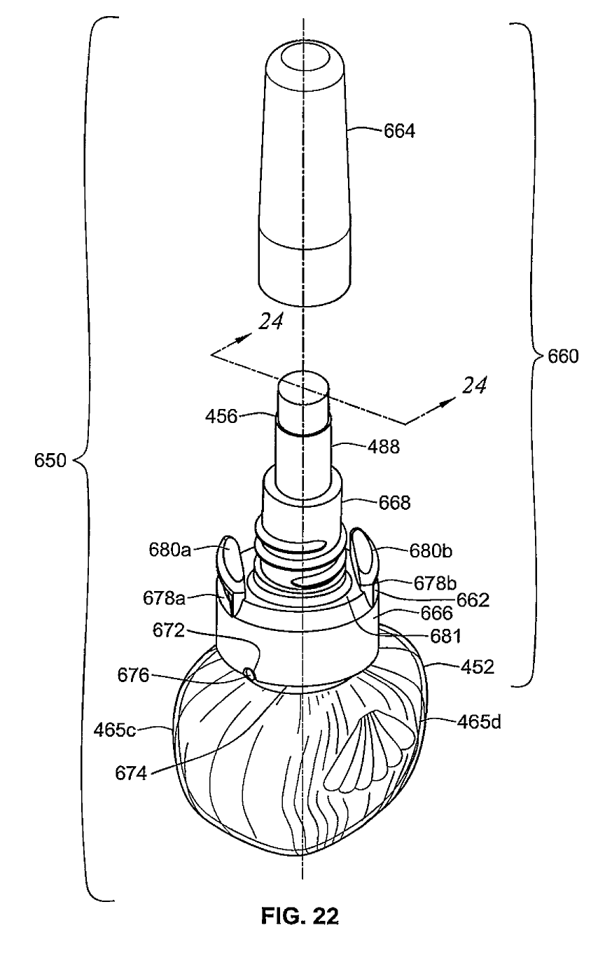

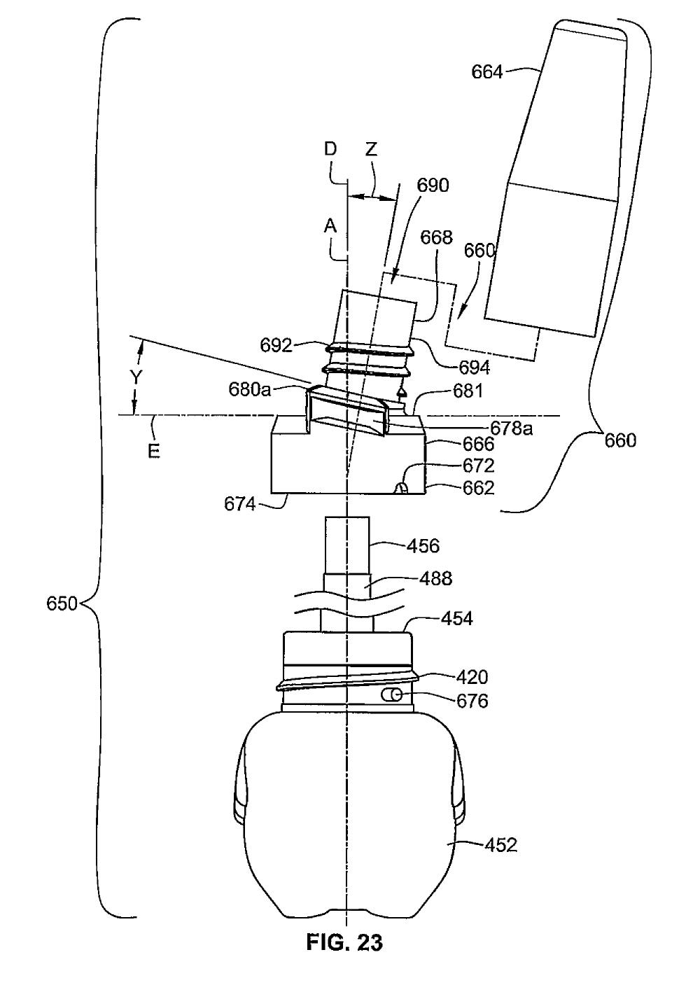

As shown in FIGS. 22-24, a seventh embodiment of a refill 650 includes a container 452, a plug assembly 454, and a wick 456 that are identical to those of the embodiment of FIGS. 16-19, and thus the reference numerals from such embodiment will be utilized for the present embodiment. Referring to FIGS. 22 and 23, the refill 650 further includes a multi-part attachment 660 having a screw-on adapter 662 and a sealing cap 664. The adapter 662 includes a lower cylindrical portion 666 and an upper angled cylindrical portion 668 extending upwardly from the lower cylindrical portion 666. The lower cylindrical portion 666 includes threads 669 on an internal surface 670 thereof that allow the adapter 662 to be screwed onto and retained by the threaded portion 470 on the neck 468 of the refill 650. The lower cylindrical portion 666 further includes a notch 672 disposed in a lower edge 674 thereof. During installation of the adapter 662 on the refill 650, the adapter 662 will only be affixed properly to the refill 650 when a cylindrical projection 676 extending outwardly from the neck 468 of the refill 650 is disposed within the notch 672. The notch 672 and projection 676 form an alignment mechanism that ensures the adapter 662 is aligned properly on the refill 650 for insertion thereof into one or more dispensers, due to the asymmetrical nature of the adapter 662. The lower cylindrical portion 666 further include upwardly extending latches 678a, 678b disposed on opposing sides thereof, wherein the latches 678a, 678b are designed to be aligned with opposing walls 465a, 465b of the refill 650. The latches 678a, 678b further include outwardly extending projections 680a, 680b that are angled with respect to a horizontal axis E that extends through defines an axial extent of a top wall 681 of the lower cylindrical portion 666. The projections 680a, 680b are preferably angled downwardly from the third wall 465c to the fourth wall 465c at the angle Y, as discussed in detail with respect to the embodiment of FIGS. 16-19.

Still referring to FIGS. 22 and 23, the upper angled cylindrical portion 668 includes a channel 690 therethrough to accommodate the wick 456, wherein the cylindrical portion 668 is disposed at the angle Z with respect to an axis A of the refill 650, wherein the angle Z is discussed in greater detail with respect to the embodiment of FIGS. 16-19. Preferably, the angles Y and Z are equal or similar to retain the cylindrical portion 668 and the axis E at about a 90 degree angle. The upper cylindrical portion 668 further includes threads 692 on an external surface 694 thereof that interact with threads (not shown) disposed on an internal surface of the sealing cap 664 to secure the sealing cap 664 to the adapter 662. Optionally, the adapter 662 may be attached to the refill 650 and/or the sealing cap 664 may be attached to the adapter 662 in any other manner know in the art including, but not limited to, a snap-fit, an interference fit, an adhesive, and the like, and combinations thereof. The adapter 662 is preferably sold affixed to the refill 650 but may alternatively be sold in a package with the refill 650 for attachment thereto. If sold separately, the adapter 662 preferably slides over and flexes the wick 656 and sheath 488 a distance enough to work in the Air Wick dispenser 120. The angle that the adapter 662 shifts the wick 656 is substantially similar to the angles discussed with respect to the embodiments of FIGS. 16-21. The cylindrical portion 668 may optionally be omitted such that the wick 456 is retained in alignment with an axis D of the attachment 660 (in a vertical position). In such case, if the wick 456 needs to be pivoted for use within one or more dispensers, the wick 456 may be pivoted by the user upon insertion, as discussed in greater detail hereinabove.

The refill 650 may be inserted into and used with both of the Glade and Air Wick dispensers 110, 120. In particular, if the refill 650 is packaged with the adapter 662 as a separate unattached piece and/or sold separately, the refill 650 alone is inserted into the Glade dispenser 110, as discussed in detail above with respect to FIG. 3. Alternatively, if the refill 650 is sold with the adapter 662 affixed thereto, the adapter 662 and sealing cap 664 are removed before inserting into the Glade dispenser 110. When the adapter 662 is removed therefrom, the wick 456 returns to its original unbiased position in which the wick 456 is generally vertical, thereby enabling insertion into the Glade dispenser 110.

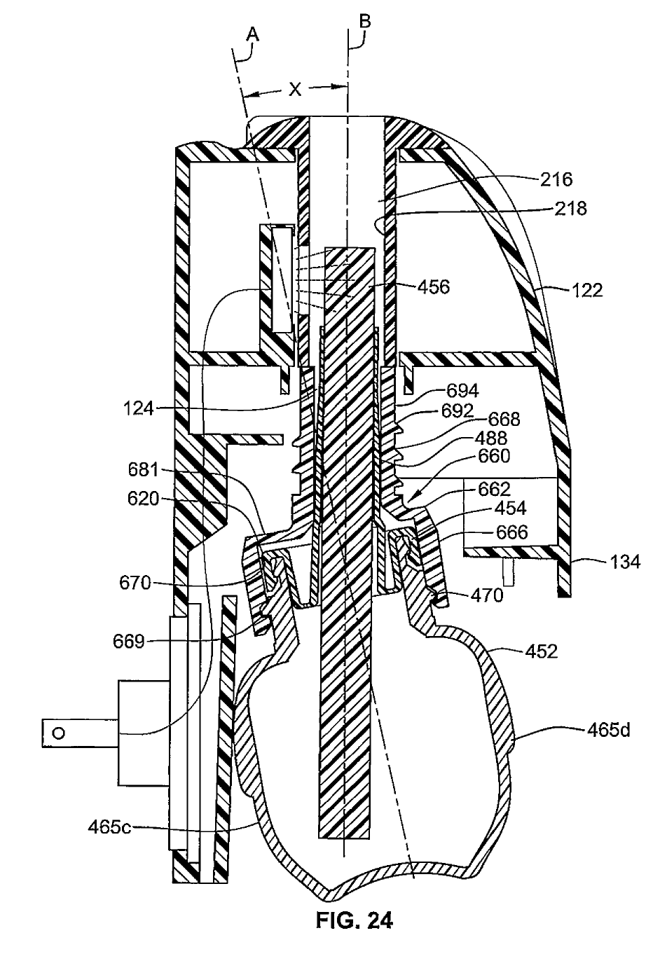

Referring to FIG. 24, if the refill 650 is packaged with the adapter 662 separate therefrom, the adapter 662 is screwed on to the refill 650, as described above, and the sealing cap 664 is removed therefrom, as described above, before insertion of the refill 650 (and adapter 662) into the Air Wick dispenser 120. Alternatively, if the refill 650 is sold with the adapter 662 affixed thereto, the sealing cap 664 is removed before use with the Air Wick dispenser 120. The user inserts the refill 650 (with adapter 662) into the dispenser 120 in a manner similar to that of the embodiments of FIGS. 16-21. As the wick 456 and upper cylindrical portion 668 of the adapter 662 enter the cavity 124 formed by the housing 122, the outwardly extending projections 680a, 680b snap over the resilient latches 136a, 136b in a manner similar to the embodiments of FIGS. 16-21 disposed within the cavity 124 of the dispenser 120 to retain the refill 650 (and adapter 662) within the dispenser 120.

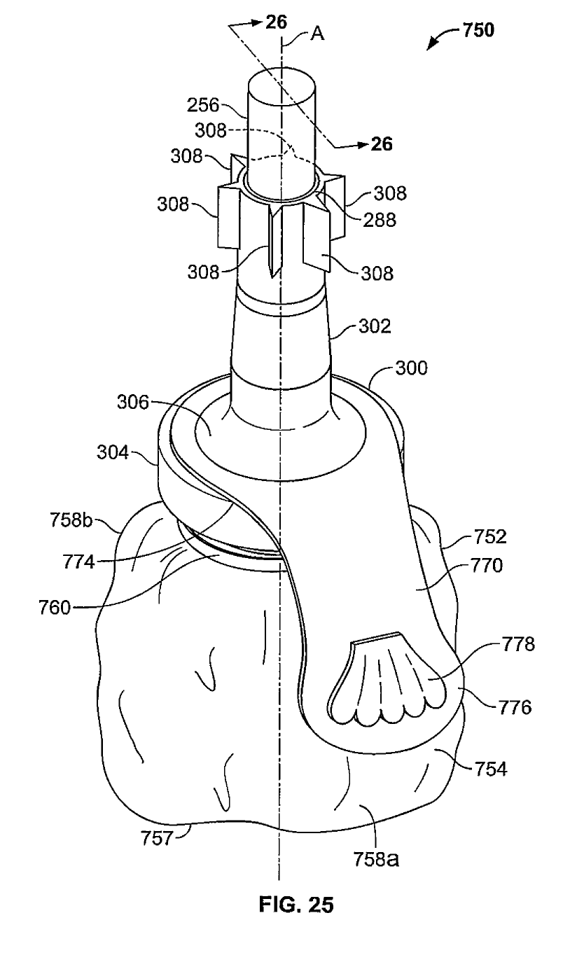

Now turning to FIGS. 25, 26, and 27, an eighth embodiment of a refill 750 comprising a container 752 and an adapter 300 is depicted, wherein the container 752 and adapter 300 are similar to previous containers and adapters with the exception of the differences noted hereinbelow. The wick 256 and plug assembly 254 are also similar to previous embodiments. The refill 750 is provided, either already attached to or in a package with, an adapter 300 that is preferably complementary in shape to and covers the sheath 288 (if present) and/or plug assembly 254 thereof. The refill 750 differs from previous embodiments in that the container 752 is made of a flexible material that is adapted to conform to interior portions of the Glade and Air Wick dispensers 110, 120 in which it is disposed, as described in more detail hereinbelow. The container 752 includes a deformable body 754 with a volatile material 756 disposed therein. The container 752 is may comprise any general shape and/or sizes so long as the material is collapsible and deformable and can conform to a dispenser in which it is disposed. The body 754 includes a base portion 757 and first and second opposing walls 758a, 758b that extend generally upwardly from the base portion 757 and are attached to a rigid neck 760. It should be noted that, although the base portion 757, the walls 758a, 758b, and other portions of the body 754 are discussed with separate reference numerals, such components typically are formed of one uniform flexible component. The flexible material of the container 752 may be made of polymers such as elastomers, low-density polyethylene, polypropylene, polystyrene, a coated cloth bag, any polymeric bag material and/or combinations thereof. The flexible material of the container 752 may comprise any material so long as the material allows deformation thereof when the container is inserted into a dispenser depending on the size and shape of the dispenser. The neck 760 of the container 752 includes a threaded portion 762 disposed on an outer surface 764 thereof and an opening 766 disposed through a top portion 768 thereof, wherein the opening 766 allows access to the volatile material 756.

Still referring to FIGS. 25, 26, and 27, the adapter 300 includes an upwardly extending hollow cylindrical portion 302 connected to an annular base 304 by a connecting portion 306. A rigid member 770 in the form of an elongate tongue extends outwardly from an edge 774 of the base 304 and/or the connecting portion 306. The rigid member 770 is adhesively attached to the base 304 and/or the connecting portion 306. Alternatively, the rigid member 770 may be attached in any suitable manner or may be integrally formed with the base 304 and/or the connecting portion 306. Still further, the rigid member 770 may be attached to any portion of the refill 750 in any way known in the art. The rigid member 770 extends downwardly from the edge 774 in an area adjacent to and substantially parallel to the first wall 758a. An end 776 of the rigid member includes a raised projection 778 extending outwardly therefrom. The projection 778 is provided in the form of an inverted shell-shaped member and is similar to the raised portion 78 described in connection with previous embodiments in form and function. The projection 778 may alternatively be in the form of any trade indicia or another shape that cooperates with a housing for insertion of the refill 750 therein.

Still referring to the adapter 300, the cylindrical portion 302 further includes ribs 308 extending outwardly therefrom similar to the ribs 196 described with respect to the embodiment of FIGS. 5-8. The number, size, and orientation of the ribs 308 may be varied as long as the ribs 308 retain the refill 750 within the Air Wick dispenser 120 while still allowing air flow through the channel 216 thereof. The adapter 300 slides over a top of the refill 750 and substantially surrounds the sheath 288 and the plug assembly 254 while allowing the wick 256 to extend upwardly out of the sheath 288 and the adapter 300, as shown in FIG. 25.

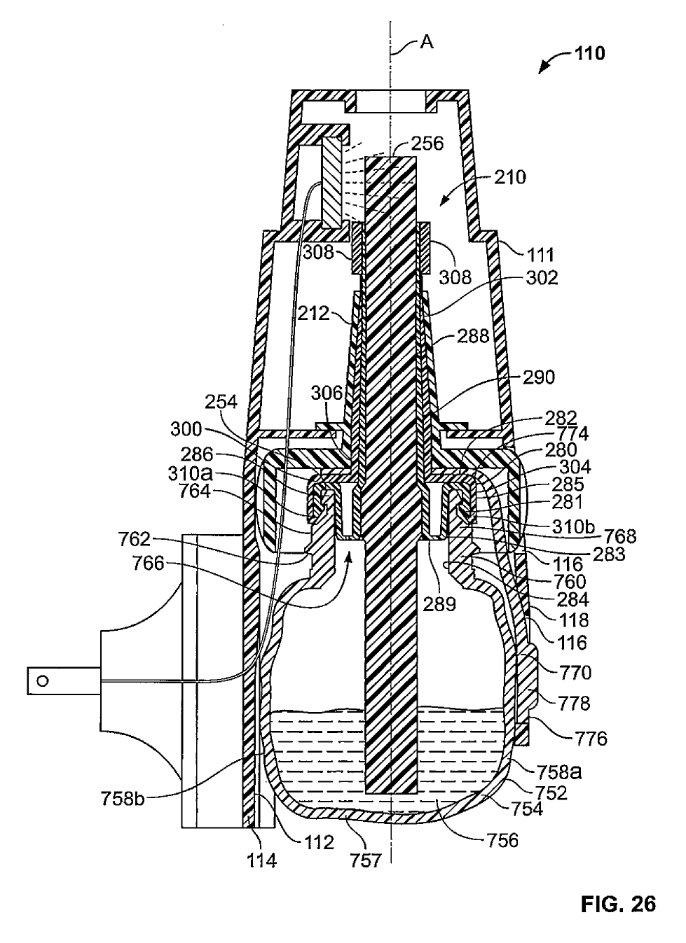

As best seen in FIG. 26, as the adapter 300 is inserted onto the sheath 288, one or more inwardly extending annular latches 310a, 310b on the adapter 300 snap over the plug assembly 254. Alternatively, as shown in a third embodiment of FIG. 12, the latches 310a, 310b on the adapter 300 may interact directly with the threaded portion 762 on the neck 760 of the container 752, for example, through a snap fit or a rotation about the threaded portion 762. Still optionally, the adapter 300 may be attached to the plug assembly 254 or the container 752 in any other manner known in the art.

The refill 750 may be inserted into and used with both the Glade and Air Wick dispensers 110, 120. Preferably, the refill 750 is sold with the adapter 300 in the same package and/or attached thereto. If the adapter 300 is not pre-attached to the refill 750, the adapter 300 is inserted onto the refill 750 in manners described previously herein prior to use. The refill 750 is inserted into the Glade dispenser 110, as shown in FIG. 26 and is retained by way of the raised projection 778 extending through the aperture 116 of the housing 111. In addition, the ribs 308 may also interact with an upper portion of the housing 111 to aid in retaining the refill 750. As the refill 750 is inserted into the housing 111, the flexible second wall 758b abuts and conforms to the groove 112 formed in the rear wall 114 of the housing 111.

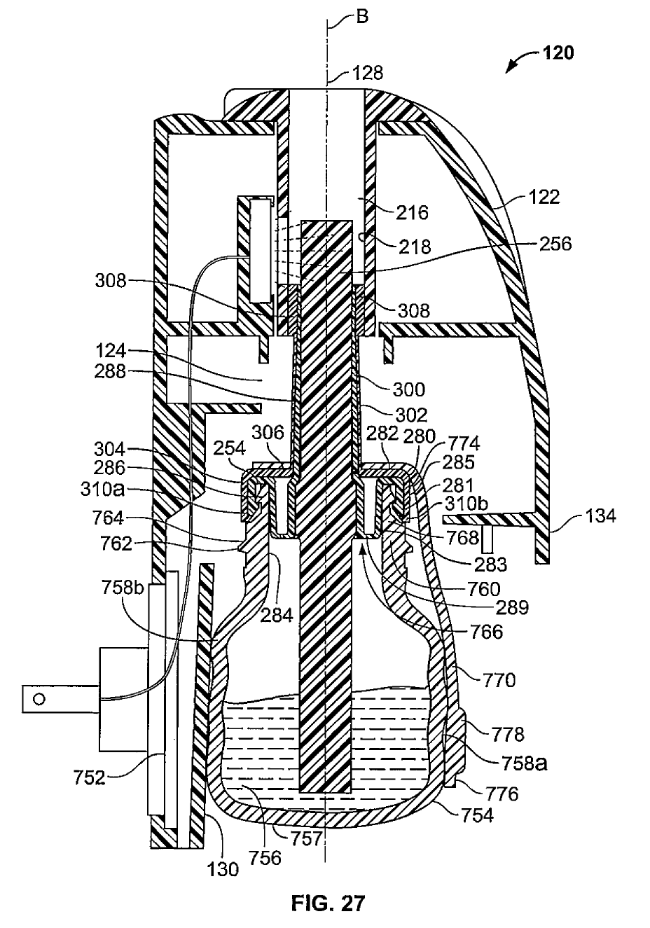

As shown in FIG. 27, the refill 750 with the adapter 300 attached thereto is inserted into the Air Wick dispenser 120 by sliding the refill 750 upwardly through the cavity 124 of the housing 122. As the refill 750 enters the cavity 124, due to the symmetrical nature of the refill 750 and the asymmetrical nature of the rear wall 130 of the dispenser 120, the flexible second wall 758b of the container 752 is pushed inwardly (and other walls of the container 752 are pushed outwardly) such that the second wall 758b conforms to the rear wall 130 to allow the refill 750 to be inserted into the dispenser 120 in a substantially upright position in a manner parallel to the centerpoint 128 of the cavity 124 as compared with previous embodiments, wherein the wick, sheath, bottle, and/or adapter are angled with respect to the centerpoint 128.

As the refill 750 is inserted into the dispenser 120, the second wall 758b of the container 752 flexes to conform to the rear wall 130 of the dispenser 120 either by way of the user angling the refill 750 upon insertion and the container 752 collapsing against the rear wall 130 once the refill 750 is retained or by way of the user inserting enough upward force on the refill 750 that the rear wall 130 pushes the second wall 758b inwardly. Regardless, once the refill 750 is secured in the Air Wick dispenser 120, the wick 256, the sheath 288, and the adapter 300 are disposed within the channel 216 and remain aligned with the axis B of the dispenser 120. Further, the ribs 308 abut the cylindrical wall 218 forming the channel 216 to retain the refill 750 within the dispenser 120 without the refill 750 engaging or being retained by the latches 136a, 136b in the Air Wick dispenser 120. Once the refill 750 is secured in the dispenser 120, at least a portion of the wick 256 is preferably exposed to allow heat from a heater in the dispenser 120 to evaporate volatile material 756 that is moved to the top portion of the wick 256 through capillary action.

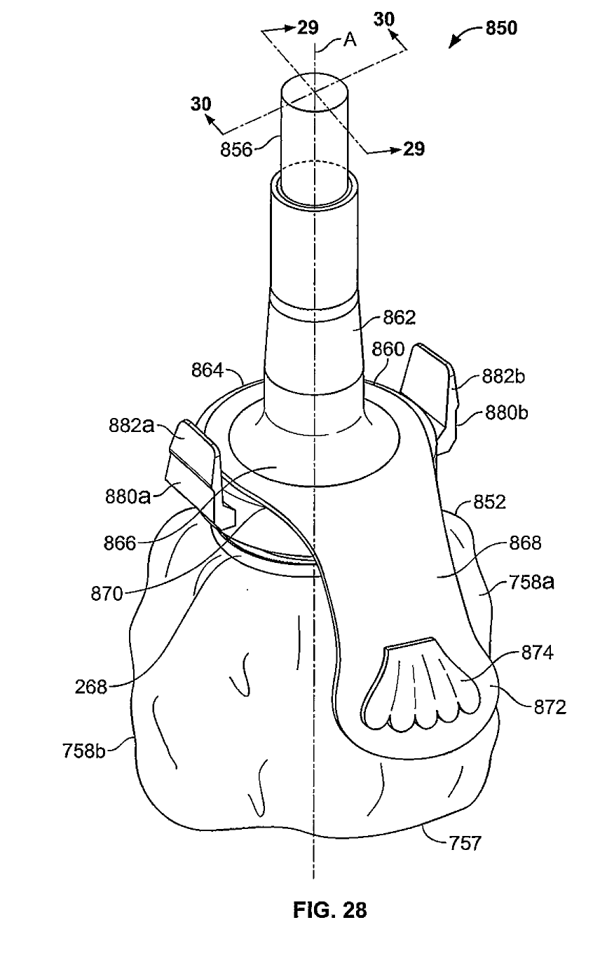

Referring now to FIGS. 28, 29, and 30, a ninth embodiment of a refill 850 for use with at least the Glade and Air Wick dispensers 110, 120 includes a flexible container 852, a plug assembly 854, and a wick 856. The container 852 is substantially identical to the container 754 of FIGS. 25, 26, and 27 in that the container 852 comprises a flexible material and does not include an integral projection extending outwardly from a front side thereof. The wick 856 is substantially identical to the wick 56 of FIG. 1. The plug assembly 854 is similar to the plug assembly described hereinabove with respect to the embodiments shown in FIGS. 9A, 9B, 10, and 11 except for the differences noted hereinbelow. For each of the components listed hereinabove that are similar to previous embodiments, like reference numerals are used.

The plug assembly 854 includes an outer annular ring 280 and an inner annular ring 281 that are generally vertical and connected by a generally horizontal surface 282. The inner annular ring 281 is designed to have an outer surface 283 that is complementary to and fits against an inner surface 284 forming an opening 274 of the container 852. The outer annular ring 280 includes an inwardly extending annular projection 285 for snapping onto an outwardly extending projection 286 on a neck 268 of the container 852. The plug assembly 854 further includes an optional sheath 288 having an inwardly extending annular portion 289 and an upwardly extending cylindrical portion 290. The annular portion 289 and the cylindrical portion 290 surround and retain the wick 856 in the same manner as discussed above with respect to FIGS. 1-3.

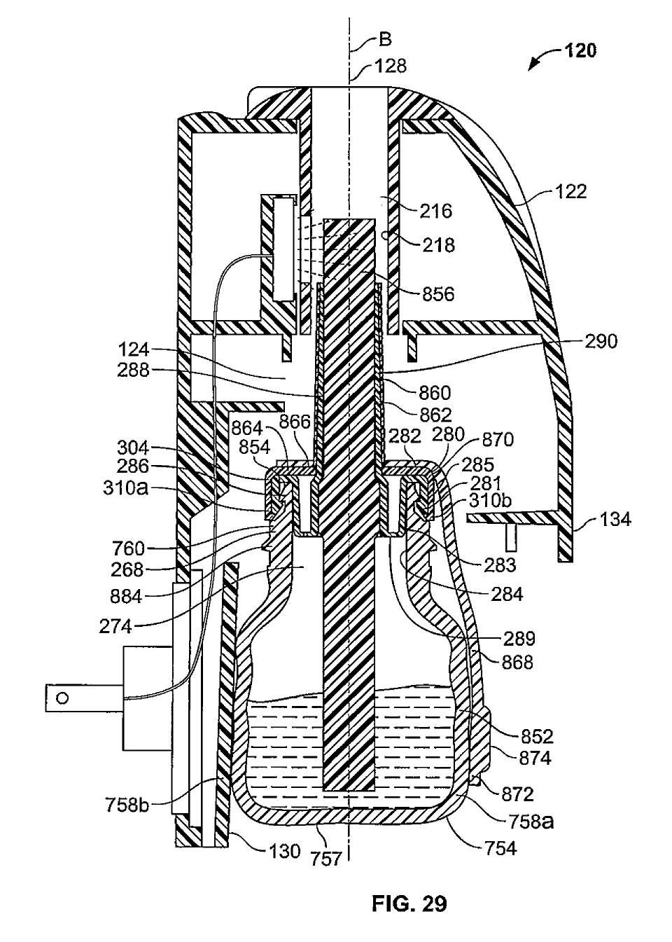

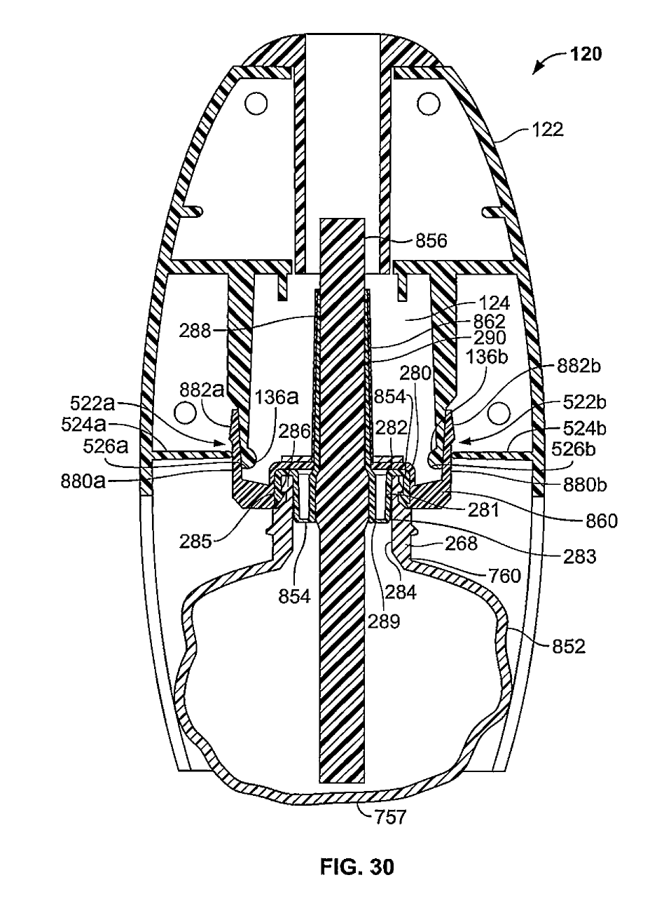

As shown best in FIGS. 29 and 30, an adapter 860 similar to the adapter 300 of the embodiment shown in FIGS. 25, 26, and 27 surrounds and covers the plug assembly 854. The adapter 860 slides over a top of the refill 850 and substantially surrounds the sheath 288 and the plug assembly 854 while allowing the wick 856 to extend upwardly out of the sheath 288 and the adapter 860, as shown in FIG. 28.

The adapter 860 includes an upwardly extending hollow cylindrical portion 862 connected to an annular base 864 by a connecting portion 866. A rigid member 868 in the form of an elongate tongue extends outwardly from an edge 870 of the base 864 and/or the connecting portion 866. The rigid member 868 is adhesively attached to the base 864 and/or the connecting portion 866. Alternatively, the rigid member 868 may be attached in any suitable manner or may be integrally formed with the base 864 and/or the connecting portion 866. Still further, the rigid member 868 may be attached to any portion of the refill 850 in any way known in the art. The rigid member 868 extends downwardly from the edge 870 in an area adjacent to and substantially parallel to the first wall 758a of the container 852. An end 872 of the rigid member includes a raised projection 874 extending outwardly therefrom. The projection 874 is provided in the form of an inverted shell-shaped member and is similar to the projection 778 described in connection with previous embodiments. The projection 874 may alternatively be in the form of trade indicia or another shape that cooperates with a housing for insertion of the refill 850 therein.

Still referring to FIGS. 28, 29, and 30, the adapter 860 further includes upwardly extending latches 880a, 880b disposed on opposing sides of the hollow cylindrical portion 862 that extends outwardly from the base 864. The latches 880a, 880b include outwardly extending projections 882a, 882b, the function of which will be discussed in greater detail hereinafter.

As the adapter 860 is inserted onto the sheath 288, one or more inwardly extending annular latches 310a, 310b on the adapter 860 snap over the plug assembly 854. Alternatively, as discussed with respect to the third embodiment of FIG. 12, the latches 310a, 310b on the adapter 860 may interact directly with a threaded portion 884 on the neck 760 of the container 852, for example, through a snap fit or a rotation about the threaded portion 884. Still optionally, the adapter 860 may be attached to the plug assembly 854 or the container 852 in any other manner known in the art.

The refill 850 may be inserted into and used with both of the Glade and Air Wick dispensers 110, 120. Preferably, the refill 850 is sold with the adapter 860 in the same package and/or attached thereto. If the adapter 860 is not pre-attached to the refill 850, the adapter 860 is inserted onto the refill 850 in manners described previously herein prior to use. The refill 850 is inserted into and retained in the dispenser 110 in the same manner as discussed with respect to FIG. 26.

The refill 850 is inserted into the Air Wick dispenser 120 by inserting the wick 856 and the sheath 288 into the cavity 124 of the housing 122 of the dispenser 120. As the refill 850 is inserted into the dispenser 120, the projections 882a, 882b extending outwardly from the latches 880a, 880b slide through gaps 522a, 522b formed between the resilient latches 136a, 136b disposed within the cavity 124 of the dispenser 122 and horizontal walls 524a, 524b aligned with bottom portions 526a, 526b of the latches 136a, 136b. In particular, the latches 880a, 880b flex inwardly (pushing the latches 136a, 136b inwardly) to allow the projections 882a, 882b to pass through the gaps 522a, 522b. Once the projections 882a, 882b pass through the gaps 522a, 522b, the latches 880a, 880b flex outwardly to prevent downward movement of the projections 882a, 882b. The refill 850 with the adapter 860 is retained in the Air Wick dispenser 120 without the refill 850 engaging or being retained by the latches 136a, 136b in the Air Wick dispenser 120. To remove the refill 850, a user must exert downward force, which forces the projections 882a, 882b downwardly against the walls 524a, 524b, thereby causing the latches 136a, 136b and 880a, 880b to move inwardly, allowing removal of the refill 850.

Although the embodiments of FIGS. 16-30 employ various types of projections for attaching the refills thereof to the Air Wick dispenser (and optionally other dispensers), any of such attachment mechanisms may be utilized for any of such embodiments.

Any of the refill embodiments herein may employ a pervious cage integral with or separate from the sheath that covers the wick, wherein the pervious cage covers a top portion of the wick. A pervious cage 120 is disclosed in greater detail in Greatbatch et al. U.S. Reissue Pat. No. 38,150, reissued on Jun. 24, 2003, the invention of which is hereby incorporated by reference herein in its entirety. One advantage of using a pervious cage in one or more of the refill embodiments herein is to protect the wick, while still allowing volatile material to be released from the wick.

Although the dispensers for which the refills herein are described as being used with are plug-in scented oil dispensers, such refills may be utilized with any type of dispenser. In particular, the dispensers employing the refills described herein may comprise a variety of forms as known in the art. The dispensers may optionally use electric power in the form of batteries or an electrical plug during the operation thereof. The dispensers also optionally may include one or more of a heater, a fan, and/or other components disposed in a housing that help facilitate the release of the volatile active. The dispensers may further include one or more openings in the housing to allow for the volatile material to be dispensed from the housing to the surrounding environment. The housing may include a variety of internal implements to help secure the various refill bottles disclosed herein, such as, for example, snaps, ridges, undercuts, lips, notches, and/or other attachment methods. The dispenser may optionally include one or more refills and may operate using a variety of timing sequences as known in the art.

Any of the embodiments described herein may be modified to include any of the structures or methodologies disclosed in connection with other embodiments.

Although the refills disclosed herein may be described as adapted for insertion and retention in the Glade and Air Wick dispensers, it is also contemplated that such refills may also be inserted into and retained in dispensers that are dimensioned differently from the Glade and Air Wick dispensers and are sold by other companies.

### INDUSTRIAL APPLICABILITY

The present invention provides a refill having a sheath that covers a portion of a wick and at least one rib extending outwardly from the sheath that is adapted to assist in retaining the refill in at least one volatile material dispenser. In particular, the refill may be inserted into a first housing in a first manner. Further, the refill may be inserted into a second differently dimensioned housing in a second, different manner.

Numerous modifications to the present invention will be apparent to those skilled in the art in view of the foregoing description. Accordingly, this description is to be construed as illustrative only and is presented for the purpose of enabling those skilled in the art to make and use the invention and to teach the best mode of carrying out same. The exclusive rights to all modifications which come within the scope of the appended claims are reserved.

We claim:

1. A volatile material refill, comprising:
a container with a volatile material disposed therein;
a plug assembly disposed within an opening of the container;
a wick retained in the container by the plug assembly;
a sheath covering a portion the wick; and
at least one rib extending outwardly from the sheath and adapted to assist in retaining the refill in at least one volatile material dispenser.

2. The refill of claim 1, further including multiple ribs extending outwardly from the sheath, wherein the ribs are generally parallel to an axial extent of the sheath and are spaced about an equal distance from each other around a circumference of an outer surface of the sheath.

3. The refill of claim 2, wherein the ribs generally extend along an upper portion of the sheath from a top edge of the sheath downwardly toward a lower portion of the sheath.

4. The refill of claim 3, wherein the ribs extend radially outwardly from the outer surface of the sheath a distance between about 0.25 mm and about 5 mm.

5. The refill of claim 4, wherein the ribs have a generally triangular cross-sectional shape along a length of the sheath.

6. The refill of claim 2, wherein a size and shape of the ribs are optimized to retain the refill within one or more dispensers while still allowing air flow through a dispenser in which the sheath and ribs are disposed.

7. The refill of claim 1, wherein the sheath is flexible to allow pivoting thereof, such that the wick may be angled with respect to a longitudinal axis of the container.

8. The refill of claim 1, wherein the container is made of a flexible material such that the container deforms to fit within at least one volatile material dispenser.

9. A wick assembly for use in a refill, comprising:
a plug assembly adapted to be inserted into and retained within a container of a refill;
a sheath extending from the plug assembly; and
at least one surface projection extending outwardly from the sheath and adapted to be disposed within a channel in one or more volatile material dispensers.

10. The wick assembly of claim 9, wherein the surface projections are in the form of ribs that are disposed on an outer surface of the sheath.

11. The wick assembly of claim 10, wherein the ribs are generally parallel to an axial extent of the sheath and are spaced about an equal distance from each other around a circumference of the outer surface.

12. The wick assembly of claim 11, wherein the ribs generally extend along an upper portion of the sheath from a top edge of the sheath downwardly toward a lower portion of the sheath.

13. The wick assembly of claim 12, wherein the ribs extend radially outwardly from the outer surface of the sheath a distance between about 0.25 mm and about 5 mm.

14. The wick assembly of claim 13, further including a wick retained by the plug assembly.

15. A method of retaining a refill in a volatile material dispenser, the method comprising the steps of:
providing a container having an elongate wick retained within the container by a plug assembly;
providing a sheath having at least two surface projections extending outwardly therefrom, wherein the sheath is disposed around at least a portion of the wick; and
allowing for positioning of the wick in a channel within a volatile material dispenser such that the surface projections contact at least one surface forming the channel.

16. The method of claim 15, further including the steps of providing the surface projections in the form of ribs.

17. The method of claim 16, wherein the ribs are generally parallel to an axial extent of the sheath and are spaced about an equal distance from each other around a circumference of an outer surface of the sheath.

18. The method of claim 17, wherein the method further includes the step of providing the sheath of flexible material such that the wick can be angled with respect to a longitudinal axis of the container.

19. The method of claim 15, wherein the method further includes the step of allowing for retention of the refill in the volatile material dispenser when the surface projections abut the surface forming the channel.

20. The method of claim 19, further including the step of providing the container of a flexible material such that the container deforms to fit within at least one volatile material dispenser.
